(19) United States
(12) Patent Application Publication
(10) Pub. No.: US 2024/0307449 A1
(43) Pub. Date: Sep. 19, 2024

(54) ORGANOID AND TUMOR-INFILTRATING LYMPHOCYTE CO-CULTURE FOR OPTIMIZATION OF PATIENT-SPECIFIC IMMUNOTHERAPY RESPONSE

(71) Applicant: The Trustees of Columbia University in the City of New York, New York, NY (US)

(72) Inventors: Alana NGUYEN, Scarsdale, NY (US); Michael M. SHEN, New York, NY (US)

(21) Appl. No.: 18/671,626

(22) Filed: May 22, 2024

Related U.S. Application Data
(63) Continuation-in-part of application No. PCT/US22/80371, filed on Nov. 22, 2022.
(60) Provisional application No. 63/282,158, filed on Nov. 22, 2021.

(51) Int. Cl. A61K 35/17; A61P 35/00; C12N 5/0783; C12N 5/09; C12Q 1/6869; G01N 33/50

(52) U.S. Cl. CPC A61K 35/17; A61P 35/00; C12N 5/0636; C12N 5/0693; C12Q 1/6869; G01N 33/5011; C12N 2501/2302; C12N 2501/51; C12N 2501/515; C12N 2502/1114; C12N 2502/1157

(57) ABSTRACT

The invention discloses a method to co-culture patient-derived tumor organoids and the corresponding patient's tumor-infiltrating lymphocytes from a tumor sample.

This application is a Continuation-in-Part under 35 U.S.C. § 111 of International Patent Application No. PCT/US2022/080371, filed on Nov. 22, 2022 which claims the benefit of and priority to U.S. Provisional Patent Application No. 63/282,158, filed on Nov. 22, 2021, the contents of each of which is hereby incorporated by reference in their entirety.

### GOVERNMENT SUPPORT

This invention was made with government support under Grant No. P01 CA221757 awarded by the National Institute of Health. The Government has certain rights in the invention.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### SUMMARY OF THE INVENTION

In some aspects, the subject matter described herein provides a method for identifying a compound for administration to a subject with cancer, the method comprising: a) producing an organoid-immune cells co-culture from a cancer tissue sample from a subject with cancer; b) contacting the organoid-immune cells co-culture with a test compound; c) determining whether growth of the organoid is inhibited in the presence of the test compound, as compared to growth of the organoid in the absence of the test compound; wherein inhibition of growth of the organoid indicates identification of the test compound for administration to said subject.

In some embodiments, the method further comprises adding cell culture media to the organoid-immune cells co-culture prior to the contacting of b), wherein the media comprises Interleukin 2 (IL2). In some embodiments, the method further comprises adding CD3/CD28 beads to the organoid-immune cells co-culture prior to the contacting of b). In some embodiments, the cell culture media comprising IL2 further comprises CD3/CD28 beads.

In some aspects, the subject matter described herein provides a method for treating cancer in a subject in need thereof, comprising: a) obtaining a cancer tissue sample from a subject with cancer; b) dissociating the sample of tissue; c) contacting the dissociated bladder tissue with a Matrigel solution and plating in a cell culture support, wherein the Matrigel solution comprises cell culture medium and Matrigel and wherein the Matrigel solution forms a matrix; d) incubating the culture of (c), wherein the dissociated tissue forms organoid-immune cells co-culture; c) contacting the organoid-immune cells co-culture with a test compound; and f) determining whether growth of the organoid is inhibited in the presence of the test compound, as compared to growth of the organoid in the absence of the test compound, wherein the test compound is administered to the subject if growth of the organoid is inhibited in the presence of the test compound.

In some embodiments, the method further comprises adding cell culture media to the organoid-immune cells co-culture prior to the contacting of e), wherein the media comprises Interleukin 2 (IL2). In some embodiments, the method further comprises adding CD3/CD28 beads to the organoid-immune cells co-culture prior to the contacting of e). In some embodiments, the cell culture media comprises IL2 further comprises CD3/CD28 beads.

In some embodiments, the cancer is a bladder cancer, a breast cancer, a prostate cancer, a pancreatic cancer, a kidney tumor, a renal cell carcinoma, a lung tumor, a colorectal cancer, uterine cancer, a thyroid tumor, or a brain cancer. In some embodiments, the cancer is bladder cancer, prostate cancer, or renal cell carcinoma. In some embodiments, the subject has an autoimmune disorder. In some embodiments, the subject is at high risk of immune adverse events.

In some embodiments, the test compound is an immunotherapy. In some embodiments, the test compound is Pembrolizumab, Nivolumab, Nivo/Ipilimumab, Durvalumab, Avelumab, Pembro/Axitinib, Nivolumab/Cabozantinib, Enfortumab Vedotin, or any combination thereof.

In some embodiments, the test compound is administered to the subject if it is determined to inhibit growth of the organoid. In some embodiments, the test compound is not administered to the subject if it is determined not to inhibit growth of the organoid.

In certain aspects the subject matter described herein provides a method for identifying a compound that inhibits cancer, the method comprising: a) producing an organoid-immune cells co-culture from a cancer tissue sample from a subject with cancer; b) contacting the organoid-immune cells co-culture with a test compound; c) determining whether growth of the organoid is inhibited in the presence of the test compound, as compared to growth of the organoid in the absence of the test compound; wherein inhibition of growth of the organoid indicates identification of a compound that inhibits cancer.

In some embodiments, the method further comprises adding cell culture media to the organoid-immune cells co-culture prior to the contacting of b), wherein the media comprises Interleukin 2 (IL2). In some embodiments, the method further comprises adding CD3/CD28 beads to the organoid-immune cells co-culture prior to the contacting of b). In some embodiments, the cell culture media comprising IL2 further comprises CD3/CD28 beads.

In certain aspects the subject matter described herein provides a method for identifying a compound that inhibits cancer, the method comprising: a) producing a library of organoid-immune cells co-cultures from cancer tissue samples from a plurality of subjects with cancer; b) contacting the organoid-immune cells co-culture library with a test compound; c) determining whether growth of one or more organoids of the library are inhibited in the presence of the test compound, as compared to growth of that organoid of the library in the absence of the test compound; wherein inhibition of growth of one or more organoids of the library indicates identification of a compound that inhibits cancer.

In some embodiments, the method further comprises adding cell culture media to the organoid-immune cells co-culture library prior to the contacting of b), wherein the media comprises Interleukin 2 (IL2). In some embodiments, the method further comprising adding CD3/CD28 beads to the organoid-immune cells co-culture library prior to the contacting of b). In some embodiments, the cell culture media comprising IL2 further comprises CD3/CD28 beads. In some embodiments, inhibition of growth of a significant number of organoids of the library indicates identification of a compound that inhibits cancer. In some embodiments, growth of at least 75% of organoids of the library indicates identification of a compound that inhibits cancer. In some embodiments, growth of at least 80% of organoids of the library indicates identification of a compound that inhibits cancer. In some embodiments, growth of at least 85% of organoids of the library indicates identification of a compound that inhibits cancer. In some embodiments, growth of at least 90% of organoids of the library indicates identification of a compound that inhibits cancer. In some embodiments, growth of at least 95% of organoids of the library indicates identification of a compound that inhibits cancer.

In some embodiments, the cancer is a bladder cancer, a breast cancer, a prostate cancer, a pancreatic cancer, a kidney tumor, a renal cell carcinoma, a lung tumor, a colorectal cancer, uterine cancer, a thyroid tumor, or a brain cancer. In some embodiments, the cancer is bladder cancer, prostate cancer, or renal cell carcinoma.

In some embodiments, the test compound is an antineoplastic agent, a chemotherapy agent, an immunotherapy, or any combination thereof. In some embodiments, the test compound is an immunotherapy. In some embodiments, the test compound is a combination of immunotherapies.

In certain aspects the subject matter described herein provides a method of identifying tumor-infiltrating lymphocytes (TILs), the method comprising: a) producing an organoid-immune cells co-culture from a cancer tissue sample from a subject with cancer; b) subjecting the organoid-immune cells co-culture to conditions wherein if tumor-infiltrating lymphocytes (TILs) are present within the organoid-immune cells co-culture they are expanded; c) subjecting the organoid-immune cells co-culture to conditions wherein if TILs are present within the organoid-immune cells co-culture they are activated; d) determining whether growth of the organoid is inhibited by the activated TILs, wherein: (i) inhibition of growth of the organoid indicates identification of TILs within the organoid-immune cells co-culture; or (ii) lack of inhibition of growth of the organoid indicates absence of TILs within the organoid-immune cells co-culture.

In certain aspects the subject matter described herein provides a method for treating cancer in a subject in need thereof, comprising: a) producing an organoid-immune cells co-culture from a cancer tissue sample from a subject with cancer; b) subjecting the organoid-immune cells co-culture to conditions wherein if tumor-infiltrating lymphocytes (TILs) are present within the organoid-immune cells co-culture they are expanded; c) subjecting the organoid-immune cells co-culture to conditions wherein if TILs are present within the organoid-immune cells co-culture they are activated; d) determining whether growth of the organoid is inhibited by the activated TILs, wherein: (i) inhibition of growth of the organoid indicates identification of TILs within the organoid-immune cells co-culture and a therapeutically effective amount of the said TILs are administered to the subject, or (ii) lack of inhibition of growth of the organoid indicates absence of TILs within the organoid and a therapeutically effective amount of TILs are not administered to the subject.

In some embodiments, steps b) and c) are performed concurrently. In some embodiments, if TILs are identified within the organoid-immune cells co-culture, the method further comprises isolating or separating TILs from the organoid-immune cells co-culture. In some embodiments, the isolated or separated TILs are expanded by culturing the cells in a cell culture medium. In some embodiments, the isolated or separated TILs are expanded by culturing the cells in a cell culture medium with allogenic irradiated PBMC feeder cells. In some embodiments, the cell culture medium is a non-Matrigel containing T-cell medium. In some embodiments, the isolated or separated TILs are expanded by culturing the cells for seven days in a mixture of TIL-CM and AIM-V medias. In some embodiments, the wherein the isolated or separated TILs are further expanded by culturing the cells for an additional seven days in AIM-V medium.

In some embodiments, if TILs are identified within the organoid-immune cells co-culture, the method further comprises isolating or separating TILs from the organoid-immune cells co-culture and expanding the isolated or separated TILs by culturing the cells in a cell culture medium before said TILs are administered to the subject.

In some embodiments, step b) comprises adding cell culture media comprising Interleukin 2 (IL2) to the organoid-immune cells co-culture. In some embodiments, step c) comprises adding CD3/CD28 beads to the organoid-immune cells co-culture. In some embodiments, the administration comprises intravenous administration of the TILs.

In some embodiments, if there is a lack of inhibition of growth of the organoid indicating absence of TILs within the organoid-immune cells co-culture, the method further comprises: c) adding a cell culture media comprising PBMCs, IL2, and CD3/CD28 beads to the organoid-immune cells co-culture; f) determining whether growth of the organoid is inhibited, wherein: (i) inhibition of growth of the organoid indicates identification of peripheral lymphocytes within the PBMCs that can target the organoid or (ii) lack of inhibition of growth of the organoid indicates absence of peripheral lymphocytes within the PBMCs that can target the organoid. In some embodiments, the PBMCs are obtained from the subject.

In some embodiments, if peripheral lymphocytes that can target the organoid are identified within the PBMCs, the method further comprises isolating or separating said peripheral lymphocytes from the organoid-immune cells co-culture. In some embodiments, the isolated or separated peripheral lymphocytes are expanded by culturing the cells in a cell culture medium. In some embodiments, the isolated or separated peripheral lymphocytes are expanded by culturing the cells in a cell culture medium with allogenic irradiated PBMC feeder cells.

In some embodiments, the cell culture medium is a non-Matrigel containing T-cell medium. In some embodiments, the isolated or separated peripheral lymphocytes are expanded by culturing the cells for seven days in a mixture of TIL-CM and AIM-V medias. In some embodiments, the wherein the isolated or separated peripheral lymphocytes are further expanded by culturing the cells for an additional seven days in AIM-V medium.

In some embodiments, if peripheral lymphocytes are identified that can target the organoid are identified within the PBMCs, the method further comprises isolating or separating said peripheral lymphocytes from the organoid-immune cells co-culture and expanding the isolated or separated peripheral lymphocytes by culturing the cells in a cell culture medium and administering said peripheral lymphocytes to the subject. In some embodiments, step b) comprises adding cell culture media comprising Interleukin 2 (IL2) to the organoid-immune cells co-culture. In some embodiments, step c) comprises adding CD3/CD28 beads to the organoid-immune cells co-culture. In some embodiments, the administration comprises intravenous administration of the peripheral lymphocytes.

In some embodiments, the cancer is a bladder cancer, a breast cancer, a prostate cancer, a pancreatic cancer, a kidney tumor, a renal cell carcinoma, a lung tumor, a colorectal cancer, uterine cancer, a thyroid tumor, or a brain cancer. In some embodiments, the cancer is bladder cancer, prostate cancer, or renal cell carcinoma. In some embodiments, producing an organoid-immune cells co-culture from a cancer tissue sample from a subject with cancer comprises: a) dissociating the sample of tissue; b) contacting the dissociated tissue with a Matrigel solution and plating in a cell culture support, wherein the Matrigel solution comprises cell culture medium and Matrigel and wherein the Matrigel solution forms a matrix; c) incubating the culture wherein the dissociated tissue forms organoid-immune cells co-culture.

In certain aspects, the subject matter described herein provides a method for producing an organoid-immune cells co-culture, the method comprising: a) obtaining a cancer tissue sample from a subject with cancer; b) dissociating the sample of tissue; c) contacting the dissociated bladder tissue with a Matrigel solution and plating in a cell culture support, wherein the Matrigel solution comprises cell culture medium and Matrigel and wherein the Matrigel solution forms a matrix; d) incubating the culture of (c) wherein the dissociated tissue forms organoid-immune cells co-cultures.

In some embodiments, the cancer is a bladder cancer, a breast cancer, a prostate cancer, a pancreatic cancer, a kidney tumor, a renal cell carcinoma, a lung tumor, a colorectal cancer, uterine cancer, a thyroid tumor, or a brain cancer. In some embodiments, the cancer is bladder cancer, prostate cancer, or renal cell carcinoma.

In some embodiments, the immune cells comprise T lymphocytes. In some embodiments, the T lymphocytes are tumor-infiltrating lymphocytes (TILs). In some embodiments, the method further comprises expanding the TILs. In some embodiments, the TILs are expanded with administration of Interleukin 2 (IL2). In some embodiments, the method further comprises activating the TILs. In some embodiments, the TILs are activated with administration of CD3/CD28 beads. In some embodiments, the expanded and activated TILs are isolated or separated from the organoids.

In some embodiments, the method further comprises: c) adding PBMCs from the subject to the organoid-immune cells co-cultures. In some embodiments, the method further comprises contacting the organoid-immune cells co-culture with Interleukin 2 (IL2). In some embodiments, the IL2 expands peripheral lymphocytes within the PBMCs that target the organoid. In some embodiments, the method further comprises contacting the organoid-immune cells co-culture with CD3/CD28 beads. In some embodiments, the CD3/CD28 beads activates peripheral lymphocytes within the PBMCs that target the organoid.

In some embodiments, the method comprises less than 40 pipetting steps. In some embodiments, the method comprises two or less centrifugation steps.

In some embodiments, if TILs are identified within the organoid-immune cells co-culture, the method further comprises sequencing the nucleic acid sequence encoding one or more T cell receptors on the surface of said TILs. In some embodiments, if peripheral lymphocytes that can target the organoid are identified within the PBMCs the method further comprises sequencing the nucleic acid sequence encoding one or more T cell receptors on the surface of said peripheral lymphocytes. In some embodiments, the nucleic acid sequence of a T cell receptor is inserted in a vector. In some embodiments, the vector is introduced into peripheral T cells isolated from the subject's PBMCs. In some embodiments, the peripheral T cells express the T cell receptor encoded by the nucleic acid sequence. In some embodiments, the peripheral T cells expressing the T cell receptor are selected for their ability to kill organoid cancer cells. In some embodiments, the selected T cells are expanded. In some embodiments, the expanded T cells are introduced back into the subject.

In some embodiments, the methods described herein further comprise evaluating cellular heterogeneity of the organoid-immune cells co-culture, or the cancer tissue sample, or both. In some embodiments, the cellular heterogeneity is evaluated using single nuclei sequencing.

In certain aspects, the subject matter described herein provides a pharmaceutical composition comprising a therapeutically effective amount of the TILs according to any method described herein.

In certain aspects, the subject matter described herein provides a pharmaceutical composition comprising a therapeutically effective amount of the peripheral lymphocytes according to any method described herein.

### BACKGROUND OF THE INVENTION

An organoid is a three-dimensional, multicellular in vitro tissue construct, which mimics its corresponding in vivo organ from which the organoid is derived. Organoids are useful in studying the corresponding in vivo organ in a controlled environment such as the tissue culture dish. Human organoids can complement existing cell culture model systems creating a more physiologically relevant setting for drug testing.

### BRIEF DESCRIPTION OF FIGURES

The patent or application file contains at least one drawing originally in color. To conform to the requirements for PCT patent application, many of the figures presented herein are black and white representations of images originally created in color. Copies of this patent or patent application publication with color drawing(s) will be provided by the United States Patent and Trademark Office upon request and payment of the necessary fee.

FIG. 1 shows a conventional organoid culture.

FIG. 2 shows a schematic of the organoid-immune cells co-culture method disclosed herein with dissociated bladder tissue. Dissociated tissue can be cultured in a conventional organoid culture or an air-liquid interface. RC=radical cystectomy. TURBT=trans urethral resection of bladder tumor.

FIGS. 3A-B show a conventional organoid culture of bladder cancer without PBMC. FIG. 3A shows that the tumor contains tumor-infiltrating lymphocytes (TILs) which are able to kill bladder tumor organoids in vitro. FIG. 3B shows that treatment of PBMC with IL2/CD3/CD28 can amplify peripheral lymphocytes even in the absence of tumor organoids. When these peripheral lymphocytes are co-cultured with tumor organoids, they also exhibit tumor killing. N=3 TIL=tumor infiltrating lymphocyte.

FIGS. 4A-B show a flow cytometry analysis of the organoid-immune cells co-culture with absolute and relative increase in CD8+ TILs in treated organoid-immune cells co-culture of bladder cancer. FIG. 4A shows composition of immune infiltration in normal bladder organoid culture: 81% are CD4+, CD8− or T-helper cells, 0.55% are CD8+, CD4− or cytotoxic T-cells, 18% are CD8−, CD4−, 4% are double positives. FIG. 4B shows lymphoid composition change with IL2 and CD3/CD28 activation. Relative decrease in CD4+, CD8− or T-helper cells; relative and absolute increase in CD8+, CD4− or cytotoxic T-cells; 5% are CD8−, CD4−; 4% are double positives.

FIG. 5 shows a flow cytometry analysis with an increase in activated CD8+ tumor-infiltrating lymphocytes in treated organoid-immune cells co-culture of bladder cancer. These CD8+ T cells have markers of degranulation and cytokine production in the organoid-immune cells co-culture, indicating that they have cytotoxic activities. Top panels show non-treated co-cultures. Bottom panels show treated co-cultures.

FIG. 6 shows a flow cytometry analysis with CD8+ memory T-cells of bladder cancer. T-lymphocytes are able to form memory subsets in culture. Effector memory T cells (Tem) CD45RA−, CD45RO+, CCR7− specializes in rapidly entering inflamed tissues. Central memory T cells (Tcm) CD45RA−, CD45RO+, CCR7+ express the chemokine receptor CCR7, allowing their re-circulation into the blood and back to lymphoid tissues. The majority of CD8+ memory cells are of the central subtype. A smaller subset of CD8+ cells are effector memory T-cells.

FIG. 7 shows a flow cytometry analysis with CD4+ Memory T-cells of bladder cancer. In unstimulated co-cultures, the majority (80%) of tumor-infiltrated CD4+ cells are CD45RO−, CCR7+ which are markers of naïve T-cells. In activated co-cultures, the majority (75%) of tumor-infiltrated CD4+ are central memory T-cells (CD45RO+, CCR7+). Effector memory T cells (Tem) (CD45RA−, CD45RO+, CCR7−) make up a smaller (21%) of CD4+ T-cells.

FIG. 8 shows conventional renal cell carcinomas (RCC) organoid culture in renal cell carcinoma without PBMCs. Addition of low-dose IL2 (300 IU) appears to be sufficient for partial tumor organoid killing in RCC co-culture. Addition with IL2 and CD3/CD28 beads kills 100% of tumor organoids.

FIG. 9 shows that there is a subset of bladder tumors from which no TILs could be amplified. Results for tumor ANB54—high-grade urothelial carcinoma (HG UC), invasive into lamina propria as an example of a bladder tumor that has no TILs. The patient's PBMC could be amplified with IL2 and CD3/CD28 beads, serving as a positive control for the culture condition. PBMCs can be amplified even though there are no TILs observed in the organoid culture.

FIG. 10 shows results for tumor ANB57-HG UC, invasive into lamina propria. Activated TILs are able to kill the tumor. However, treatment with checkpoint inhibitor Pembrolizumab is not sufficient to activate the TILs to kill the tumor organoids.

FIG. 11 shows results for tumor ANRN2-tumor adjacent organoids. Tumor adjacent or non-tumor tissues can grow as organoids and survive passaging. P1 and P2 of ANRN2 stock are frozen down. Immunofluorescence images show that the tissue is indeed normal kidney parenchyma with CD10+ and CK7+.

FIG. 12 shows results for tumor JOKT6 and tumor-adjacent JOKN6. T-cells killed tumor organoids but not normal adjacent tissues. Sample is from a radical nephrectomy of a 10 cm mass, showing ccRCC with extension into the renal sinus and perinephric tissue. Final path pT3aNx. JOKT6, and JOKN6, expanded and have been frozen down, along with TILs and PBMC.

FIG. 13 shows organoid cell death measured by a TUNEL assay. TUNEL staining indicates dying cells. CK5 and CK8 are luminal cell markers.

FIG. 14 shows Granzyme B and TUNEL staining on untreated organoid co-culture and activated (IL2 and CD3/CD28 beads) co-culture. Granzyme B indicates cytotoxic T cell release. Granzyme B expression is increased in treated culture compared to Pembrolizumab/IL2 treatment or untreated control. Increased tumor cell death is observed with activation of TILs.

FIG. 15 shows that organoid-immune cells co-culture can be used to test anti-PD1 treatment with Pembrolizumab in vitro. Tumor ANB55 is an immunoresponsive organoid. Tumor organoids had a response to PD1 blockade. Activated tumor organoid immune co-culture is treated with IL2 and CD3/CD28 beads and serves as positive control. PATIENT: 70M presented with 2-3 cm mass on cystoscopy. Patient had TURBT on Apr. 12, 2021 showing HG UC and re-TURBT was negative. BCG induction to be initiated in June 2021. PATHOLOGY: Sample from Apr. 12, 2021 TURBT showed HG papillary urothelial carcinoma, with lamina propria invasion. Re-TURBT in May 2021 was negative for carcinoma. The tumor organoid responds to Pembrolizumab treatment. Cell killing has begun in the IL2/Pembrolizumab condition. Complete killing of tumor cells is observed in the activated TILs condition.

FIG. 16 shows that the patient has tumor-specific T-cells that are present in blood but not in primary tumor. Tumor ANB60 is an immune-non-responsive organoid. Tumor organoids did not respond to PD1 blockade or IL2/beads and only partially responds to IL2 and CD3/CD28 beads. Addition of PBMC in activated condition with IL2 and CD3/CD28 beads demonstrates that peripheral lymphocytes can be “trained” to recognize and kill tumors in this instance, more effective than TILs. PATIENT: 66F presented with gross hematuria several years ago, recommended cysto but never had it. Developed gross hematuria and abdominal pain in May 2021. PATHOLOGY: Sample from May 17, 2021 TURBT showed non-invasive LG papillary urothelial carcinoma.

FIG. 17 shows that organoid-immune cells co-culture can be adopted for treatment of RCCs, and both the tumor organoids and the T-cells can be passaged.

FIGS. 18A-B shows that the organoid-immune cells co-culture disclosed herein can be adopted for castration-resistant prostate cancer. FIG. 18A shows tumor ANP2—a single mCRPC sample. Activation is achieved with treatment with IL2 and CD3/CD28 beads. This mCRPC tumor does not respond to checkpoint blockade in vitro, and it also does not have amplified TILs. However, when PBMC is added and activated with IL2 and CD3/CD28 beads, there are tumor-specific lymphocytes and can kill tumor organoids. FIG. 18B shows a summary of the organoid-immune cells co-cultures.

FIGS. 19A-B shows tumor ANB45 NMIBC (papillary-urothelia carcinoma (UC), low grade (LG) and high grade (HG)). FIG. 19A shows Vectra staining. FIG. 19B shows air-liquid interphase culture on left and organoid culture on right.

FIG. 20 shows tumor ANB55, NMIBC (HG papillary UC, lamina propria invasion), ANB55 P0, Pembro ANB55 P0.

FIG. 21 shows tumor ANB57—high-grade papillary urothelial carcinoma, with laminar propria invasion.

FIG. 22 shows tumor ANB58—upper tract non-invasive (pTa) urothelial carcinoma, Low grade papillary tumor.

FIG. 23 shows quality control and performance metrics for multiplexed single nuclei sequencing. The majority of nuclei sequenced came from a single cell.

FIG. 24 shows quality control statistics for single nuclei sequencing experiments. Nuclei with >20% mitochondrial genes (MT genes) and nuclei with less than 1500 UMIs (unique molecular identifiers) were filtered out.

FIG. 25 shows that the single nuclei sequencing disclosed herein identifies multiple subpopulations of cells using singleR references.

FIG. 26 shows unsupervised clustering of all nuclei after batch correction.

FIG. 27A-B shows clustering of all single nuclei combined. FIG. 27A shows unsupervised clustering of all nuclei after batch correction. FIG. 27B shows the different cell types per cluster in percentages. Distinct cell lineages which can be identified are epithelial and malignant cells (C0-C1-C2, show in blue), primarily cancer associated fibroblasts (C3, shown in green), immune cells of lymphoid and myeloid lineages (C4, shown in purple), and endothelial cells (C5, shown in orange).

FIG. 28 shows the composition of cells in original parental tumors. Most are enriched in tumor cells and cancer-associated fibroblasts. Distinct cell lineages which can be identified are epithelial and malignant cells (top rectangle, show in blue), cancer associated fibroblasts (2^{nd} rectangle from the top, shown in green), immune cells of lymphoid and myeloid lineages (3^{rd} rectangle from the top, shown in purple), and endothelial cells (bottom rectangle, shown in orange).

FIG. 29 shows the single nuclei sequencing and cell annotations for tumor sample ANB55. Within the immune compartment, CD4+ T cells, macrophages, and monocytes could be detected but not CD8+ T cells.

FIG. 30 shows the single nuclei sequencing and cell annotations for tumor sample ANB60. Within the immune compartment, CD4+ T cells, macrophages, and monocytes could be detected but not CD8+ T cells.

FIG. 31 shows the single nuclei sequencing and cell annotations for tumor sample ANB64. Within the immune compartment, CD4+ T cells, macrophages, and monocytes could be detected but not CD8+ T cells.

FIG. 32 shows the single nuclei sequencing and cell annotations for tumor sample ANB80 and ANB76. Within the immune compartment, CD4+ T cells, macrophages, and monocytes could be detected but not CD8+ T cells.

FIG. 33 shows the single nuclei sequencing and cell annotations for tumor sample JOB 51.2. Within the immune compartment, CD4+ T cells, macrophages, and monocytes could be detected but not CD8+ T cells.

FIG. 34 shows the single nuclei sequencing and cell annotations for tumor sample ANB84. Within the immune compartment, CD4+ T cells, macrophages, and monocytes could be detected but not CD8+ T cells.

FIG. 35 shows viability assays of tumor ANB55 incubated with various treatments.

FIG. 36 shows viability assays of tumor JOB51.2 incubated with various treatments.

FIG. 37 shows viability assays of tumor ANB63 incubated with various treatments.

FIG. 38 shows viability assays of tumor ANB74 incubated with various treatments.

### DETAILED DESCRIPTION OF THE INVENTION

Tumor-infiltrating lymphocytes (TILs) are a type of cell therapy that utilize lymphocytes recognizing an individual patient's tumor. Currently, TILs are isolated and identified by growing individual lymphocytes cultures separately before assaying for tumor recognition. Current methods to test tumor recognition do not test for TIL efficacy or select for tumor-killing ability, increasing the likelihood treatment will not effectively target the tumor cells

T-cells are broad class of immune cell that protect the body against infection and can kill cancer cells. Tumor-infiltrating lymphocytes (TILs) are T-lymphocytes with higher immunological reactivity towards tumor cells than host cells (Badalamenti G, Fanale D, Incorvaia L, Barraco N, Listì A, Maragliano R, Vincenzi B, Calò V, Iovanna J L, Bazan V, Russo A. Role of tumor-infiltrating lymphocytes in patients with solid tumors: Can a drop dig a stone? Cell Immunol. 2019 September; 343:103753.1). Tumor-infiltrating lymphocytes can be isolated and amplified from patients before re-introduction as a cell therapy to target tumor cells (Lizée G, Overwijk W W, Radvanyi L, Gao J, Sharma P, Hwu P. Harnessing the power of the immune system to target cancer. Annu Rev Med. 2013; 64: pp. 71-90). TILs are composed of T cells with heterogeneous T-cell receptor clones recognizing a broad array of tumor antigens and is more suitable for targeting tumors with high heterogeneity or mutational burden (Wang S, Sun J, Chen K, Ma P, Lei Q, Xing S, Cao Z, Sun S, Yu Z, Liu Y, Li N. Perspectives of tumor-infiltrating lymphocyte treatment in solid tumors. BMC Medicine. 2021 June; 19: 140). TILs are currently being investigated for the treatment of solid tumors, such as melanoma, renal cell carcinoma, and breast cancer (Wang S. et al.). Checkpoint blockade therapies (such as anti-PD1 or anti-CTLA4 antibodies) block the ability of tumor cells to inhibit immune cell function (Ribas A, Wolchok JD. Cancer immunotherapy using checkpoint blockade. Science. 2018; 359(6382): pp. 1350-1355).

Tumor-infiltrating lymphocytes (TILs) can be grown from primary tumors using various approaches, and re-introduced into patients as a form of adoptive cell transfer. The standard in the field is that prior to their re-introduction into patients, these cells were neither tested nor selected for their efficacy and specificity ex vivo. In addition, we have previously established a culture system to grow tumor organoids from primary patient bladder tumor samples, and have shown that these three-dimensional models recapitulate properties of their corresponding parental tumors. To date, however, it has been difficult to demonstrate that TILs can co-exist in culture with patient-derived tumor organoids and display functional activities.

In some aspects, the subject matter described herein relates to a system for co-culturing of patient-derived tumor organoids with the corresponding patient's tumor-infiltrating lymphocytes from a small tumor sample (˜3 mm). In some embodiments, the tumors are isolated from a patient at the time of initial biopsy or surgical resection of the bladder, kidney, or prostate. The tumors can then be digested and grown in a matrix-like environment as described herein. Tumor-infiltrating lymphocytes are amplified using a combination of IL2 and beads expressing markers that mimic antigen-presentation. In some aspects, the subject matter described herein relates to activating lymphocytes to specifically kill tumor organoids, sparing the normal adjacent tissues. In some embodiments, the co-culture system described herein can be used to activate patient lymphocytes to specifically target tumor cells. In some embodiments, the activated lymphocytes can be re-introduced into the patient as a form of adoptive cell therapy. In some embodiments, the co-culture system itself can be used to test immunotherapy such as anti-PD1 inhibitors (e.g., Pembrolizumab) to test for therapy efficacy before the therapy is given to the patient. This will allow for only effective therapies to be administered to patients, decreasing side effects and time wasted on treatment with ineffective methods.

In some embodiments, the system described herein can be used for pre-treatment screening for patients who would benefit from immunotherapy. The response rate to first line immunotherapy in bladder cancer is approximately 24% (disease control rate 47%) in the metastatic setting (Balar A V, Castellano D, O'Donnell P H, Grivas P, Vuky J, Powles T, Plimack E R, Hahn N M, de Wit R, Pang L, Savage M J, Perini R F, Keefe S M, Bajorin D, Bellmunt J. First-line pembrolizumab in cisplatin-ineligible patients with locally advanced and unresectable or metastatic urothelial cancer (KEYNOTE-052): a multicentre, single-arm, phase 2 study. Lancet Oncol. 2017 November; 18(11): 1483-1492. doi: 10.1016/S1470-2045(17)30616-2. Epub 2017 Sep. 26. PMID: 28967485). The response rate to a-PD1 monotherapy in RCC is slightly better at ˜35% (disease control rate 58%) in the metastatic setting (McDermott D F, Lee J L, Bjarnason G A, Larkin J M G, Gafanov R A, Kochenderfer M D, Jensen N V, Donskov F, Malik J, Poprach A, Tykodi S S, Alonso-Gordoa T, Cho D C, Geertsen P F, Climent Duran M A, DiSimone C, Silverman R K, Perini R F, Schloss C, Atkins M B. Open-Label, Single-Arm Phase II Study of Pembrolizumab Monotherapy as First-Line Therapy in Patients With Advanced Clear Cell Renal Cell Carcinoma. J Clin Oncol. 2021 Mar. 20; 39(9):1020-1028. doi: 10.1200/JCO.20.02363. Epub 2021 Feb. 2. PMID: 33529051; PMCID: PMC8078336), which increases with the addition of a tyrosine kinase inhibitor such as Axitinib (Rini B I, Plimack E R, Stus V, Gafanov R, Hawkins R, Nosov D, Pouliot F, Alekseev B, Soulières D, Melichar B, Vynnychenko I, Kryzhanivska A, Bondarenko I, Azevedo S J, Borchiellini D, Szczylik C, Markus M, McDermott R S, Bedke J, Tartas S, Chang Y H, Tamada S, Shou Q, Perini R F, Chen M, Atkins M B, Powles T; KEYNOTE-426 Investigators. Pembrolizumab plus Axitinib versus Sunitinib for Advanced Renal-Cell Carcinoma. N Engl J Med. 2019 Mar. 21; 380(12): 1116-1127. doi: 10.1056/NEJMoa1816714. Epub 2019 Feb. 16. PMID: 30779529). Therefore, approximately 50% of patients do not derive any benefit from immunotherapy in the first line setting, and this number goes down in the second and third line. Furthermore, the treatment is expensive (˜$100K/yr) and approximately 15% of patients experience Gr. 3 or higher adverse events, some of which are irreversible and require lifelong treatment. Thus, accurate prediction of which patient will benefit will be important for the following reasons: (1) allow patients to move onto treatments from which they might derive benefit, (2) avoid a costly treatment that has no benefit, and (3) avoid immune adverse reactions. In some embodiments, the subject matter disclosed herein relates to an organoid-tumor infiltrating lymphocyte co-culture system that can be used to test response to immunotherapy such as Pembrolizumab for both renal cell carcinoma (RCC) and bladder. This system can be used in a “personalized immunotherapy” approach for patients for which the benefit might not be clear, but the patient is at risk for adverse reactions such as patients with autoimmune disorders. In some embodiments, the system of growing organoids can allow high-throughput screening, and can then be applied to the general cancer population.

In some embodiments, the system described herein can be used for optimization of T-cells for re-introduction into patient as a form of T-cell therapy. T-cell therapy for cancer is a promising but costly approach to treating cancer. For example, the estimated CAR-T therapy which includes harvesting, amplification, genetic engineering, and reintroduction can cost upwards of $300K per patient. These T-cells are derived from peripheral blood and therefore might have not seen any tumors. In some embodiments, the subject matter described herein relates to systematically isolating and amplifying tumor-specific infiltrating lymphocytes from a very small tumor sample (˜3 mm tumor). These T-cells can specifically kill the organoids with which they co-exist but not the adjacent tissues. The T-cells that can successfully kill tumors. The T-cells can therefore be amplified and re-introduced back into the patient as a form of T-cell therapy. The benefit to this system over an engineered system is as follows: (1) reduced cost by eliminating the genetic engineering step, (2) TILs are tumor specific and therefore have already been primed to kill tumor cells. The advantage of the organoid system described herein is the ability to test the T-cells' ability to kill organoids prior to introducing them back into the patient. If the amplified TILs are unable to kill organoids, then they might not work in the patient, thereby sparing the patient an ineffective treatment, adverse events, and costs associated with it. Currently, no cell-based therapy is known to work until it is infused into the patient and there are instances where the patients have adverse events without real clinical benefit. The advantage of our system to test efficacy and select for tumor-killing ability before the therapies are given to the patient is particularly important for T-cell therapies such as TILs therapy or CAR-T therapy as they are known to cause severe adverse effects such as cytokine release syndrome. The ability to select for tumor-specific T-cells means that we can potentially increase effectiveness of cell-based therapy while reducing adverse events.

In some embodiments, the system described herein can be used for testing of immunotherapy agents that would give patients the best response. New and emerging immunotherapy approaches are being routinely tested in early-phase clinical trials, often based on their efficacy in animal models of cancer. In some embodiments, the subject matter described herein relates to an organoid-tumor infiltrating lymphocyte system that can improve the process of immunotherapy development by improving pre-clinical testing, and therefore would be cost-saving for pharmaceutical companies and other commercial entities trying to bring a particular product to the market. In a subset of tumors, there are amplified TILs without tumor killing ability. These tumors, in particular, would be useful for testing new immunotherapies because they suggest that the lymphocytes are present but there are inhibitory mechanisms preventing them from being activated or targeting the tumors efficiently.

In some embodiments, the subject matter described herein relates to screening for immunotherapy sensitivity. Small biopsies from tumors can be taken at patient diagnosis for organoid-tumor infiltrating lymphocyte co-culture to test against a variety of currently available immunotherapies and their various combinations. These immunotherapies include but are not limited to Pembrolizumab, Nivolumab, Nivo/Ipilimumab, Durvalumab, Avelumab, Pembro/Axitinib, Nivolumab/Cabozantinib, Enfortumab Vedotin, etc. Based on the effect of these agents on tumor organoid killing or proliferation, specific recommendations can be made for each patient. Current predictive methods include CPS (combined proportional score), TPS (tumor proportional score), and TMB (tumor mutational burden), but none of these methods are direct predictors of efficacy. There are patients with high CPS/TMB with no response to immunotherapy and patients with low CPS/TMB with remarkable response to immunotherapy, suggesting that multiple factors are affecting the immune system's ability to specifically target cancer cells. The system described herein allows for the native tumor to be taken and for specific agents to be tested on their ability to stimulate killing. Given the minimal manipulation involved, the testing method described herein can be offered to specific subsets of patients, such as patients at high risk for immune adverse effects or patients for whom no other options are available.

In some embodiments, the subject matter described herein relates to T-cell therapy. The tumor-infiltrating lymphocytes described herein can be consistently expanded in the co-culture system and T-cells that specifically kill tumor cells can be selected. This system not only amplifies tumor-specific T-cells but can also be predictive of whether the T-cells will be able to kill the tumor. This system represents a specific method for selecting tumor-infiltrating lymphocytes with the expressed purpose of infusing them back into the patient.

In some embodiments, the subject matter described herein relates to the testing of new immunotherapy agents. The testing of therapies would be geared towards drug development. The limiting factor is the collection of a large enough sample size for meaningful interpretation of the data.

In some embodiments, the methods described herein further comprise evaluating cellular heterogeneity of the organoid-immune cells co-culture. In some embodiments, the methods described herein further comprise evaluating cellular heterogeneity of the cancer tissue sample. In some embodiments, the methods described herein further comprise evaluating cellular heterogeneity of the organoid-immune cells co-culture and the cancer tissue sample. Cellular heterogeneity can be evaluated using techniques known in the art, such as, but not limited to, sequencing techniques such as single-cell sequencing or single nuclei sequencing. In some embodiments, the cellular heterogeneity is evaluated using single nuclei sequencing.

In some embodiments, the evaluation of cellular heterogeneity comprises lysing cells of a sample; generating a single nuclei suspension; and performing single nuclei sequencing. In some embodiments, a sample can be a tumor biopsy from the subject (e.g., a cancer tissue sample). In some embodiments, a sample can be an organoid-immune cell co-culture. In some embodiments, the sequencing results can be annotated using a reference dataset to identify cells within the sample. In some embodiments, the sequencing results can be annotated to identify immune cells within the sample. In some embodiments, the identified cell types can be used to design new studies using the organoid-immune cells co-culture described herein. In some embodiments, lysing cells of a sample comprises lysing the sample on ice in lysis buffer. In some embodiments, the sample is a frozen sample. In some embodiments, lysis is stopped by diluting the lysed sample in buffer (e.g. PBS). In some embodiments, the single nuclei suspension is generated by precipitating and filtering the lysed sample. In some embodiments, the nuclei quality of the single nuclei suspension is evaluated by evaluating nuclear morphology. In some embodiments, the method of evaluating cellular heterogeneity is continued only for samples wherein the single nuclei suspension comprises nuclei of high quality. In some embodiments, nuclei of the single nuclei suspension are tagged with an antibody. In some embodiments, nuclei of the single nuclei suspension are tagged with an anti-nucleoporin antibody.

In some embodiments, the evaluation of cellular heterogeneity can be performed for two or more samples simultaneously. In such an embodiment, evaluation of cellular heterogeneity comprises lysing cells of each sample; generating a single nuclei suspension for each sample; tagging nuclei of the single cell suspension for each sample with a unique antibody; pooling the single nuclei suspensions of all samples; and performing single nuclei sequencing. In some embodiments, a sample can be a tumor biopsy from the subject (e.g., a cancer tissue sample). In some embodiments, a sample can be an organoid-immune cell co-culture. In some embodiments, the sequencing results can be annotated using a reference dataset to identify cells within the sample. In some embodiments, the sequencing results can be annotated to identify immune cells within the sample. In some embodiments, the identified targets can be used to design new studies using the organoid-immune cells co-culture described herein. In some embodiments, lysing cells of a sample comprises lysing each sample on ice in lysis buffer. In some embodiments, the sample is a frozen sample. In some embodiments, lysis of each sample is stopped by diluting the lysed sample in buffer (e.g. PBS). In some embodiments, the single nuclei suspension is generated by precipitating and filtering the lysed sample. In some embodiments, the nuclei quality of the single nuclei suspension for each sample is evaluated by evaluating nuclear morphology. In some embodiments, the method of evaluating cellular heterogeneity is continued only for samples wherein the single nuclei suspension comprises nuclei of high quality. In some embodiments, the nuclei of the single nuclei suspension for each sample are tagged with a unique anti-nucleoporin antibody. In some embodiments, the single nuclei suspensions are pooled and counted.

### Organoid-Immune Cells Co-Culture and Methods of Producing

In certain aspects, the subject matter described herein provides a method for producing an organoid-immune cells co-culture from a cancer tissue sample from a subject with cancer, the method comprising: a) dissociating the sample of tissue; b) contacting the dissociated tissue with a Matrigel solution and plating in a cell culture support, wherein the Matrigel solution comprises cell culture medium and Matrigel and wherein the Matrigel solution forms a matrix; c) incubating the culture wherein the dissociated tissue forms organoid-immune cells co-culture.

In certain aspects, the subject matter described herein provides a method for producing an organoid-immune cells co-culture, the method comprising: a) obtaining a cancer tissue sample from a subject with cancer; b) dissociating the sample of tissue; c) contacting the dissociated bladder tissue with a Matrigel solution and plating in a cell culture support, wherein the Matrigel solution comprises cell culture medium and Matrigel and wherein the Matrigel solution forms a matrix; d) incubating the culture of (c) wherein the dissociated tissue forms organoid-immune cells co-cultures.

In some embodiments, the cancer is a bladder cancer, a breast cancer, a prostate cancer, a pancreatic cancer, a kidney tumor, a renal cell carcinoma, a lung tumor, a colorectal cancer, uterine cancer, a thyroid tumor, or a brain cancer. In some embodiments, the cancer is bladder cancer, prostate cancer, or renal cell carcinoma.

In some embodiments, the immune cells comprise T lymphocytes. In some embodiments, the T lymphocytes are tumor-infiltrating lymphocytes (TILs). In some embodiments, the method further comprises expanding the TILs. In some embodiments, the TILs are expanded with administration of Interleukin 2 (IL2). In some embodiments, the method further comprises activating the TILs. In some embodiments, the TILs are activated with administration of CD3/CD28 beads.

In some embodiments, the expanded and activated TILs are isolated or separated from the organoids. In some embodiments, the method further comprises: c) adding PBMCs from the subject to the organoid-immune cells co-cultures. In some embodiments, method further comprises contacting the organoid-immune cells co-culture with Interleukin 2 (IL2). In some embodiments, the IL2 expands peripheral lymphocytes within the PBMCs that target the organoid. In some embodiments, method further comprises contacting the organoid-immune cells co-culture with CD3/CD28 beads. In some embodiments, the CD3/CD28 beads activates peripheral lymphocytes within the PBMCs that target the organoid.

In some embodiments, the method comprises less than 40 pipetting steps. In some embodiments, the method comprises two or less centrifugation steps.

In some embodiments, the subject matter described herein relates to a method for co-culturing of patient-derived tumor organoids with the corresponding patient's immune cells. In some embodiments, the immune cells are T lymphocytes. In some embodiments the T lymphocytes are tumor-infiltrating lymphocytes (TILs). In some embodiments, the co-culture is derived from a small tumor sample. In some embodiments, the tumor sample is ˜3 mm. In some embodiments, the tumors are isolated from a patient. In some embodiments, the patient is a cancer patient. In some embodiments, the tumors are isolated from the patient at the time of initial biopsy or surgical resection. In some embodiments the initial biopsy or surgical resection is of the bladder. In some embodiments the initial biopsy or surgical resection is of the kidney. In some embodiments the initial biopsy or surgical resection is of the breast, the prostate, the pancreas, the lung, the colon, the uterus, the thyroid, or the brain.

In some embodiments, the isolated tumors are further processed for culturing while they are still fresh. In some embodiments, the isolated tumors are frozen. In some embodiments, the isolated tumors are preserved in a paraffin block. In some embodiments, the isolated tumors are formalin-fixed, paraffin-embedded (FFPE) in blocks. In some embodiments, the isolated tumors are stored in any suitable conditions known in the art prior to further processing.

In some, embodiments, the tumors are further processed for culturing by mechanical dissociation. In some embodiments, the tumors are minced. In some embodiments, the tumors are minced with scissors. In some embodiments, the tumors are chemically dissociated. In some embodiments, the tumors are chemically dissociated with hyaluronidase and/or collagenase. In some embodiments, the tumors are digested. In some embodiments, the tumors are minced prior to digestion. In some embodiments, the tumors are digested with one or more proteases. In some embodiments, the protease is trypsin. In some embodiments, the tumors are digested with any suitable protease known in the art.

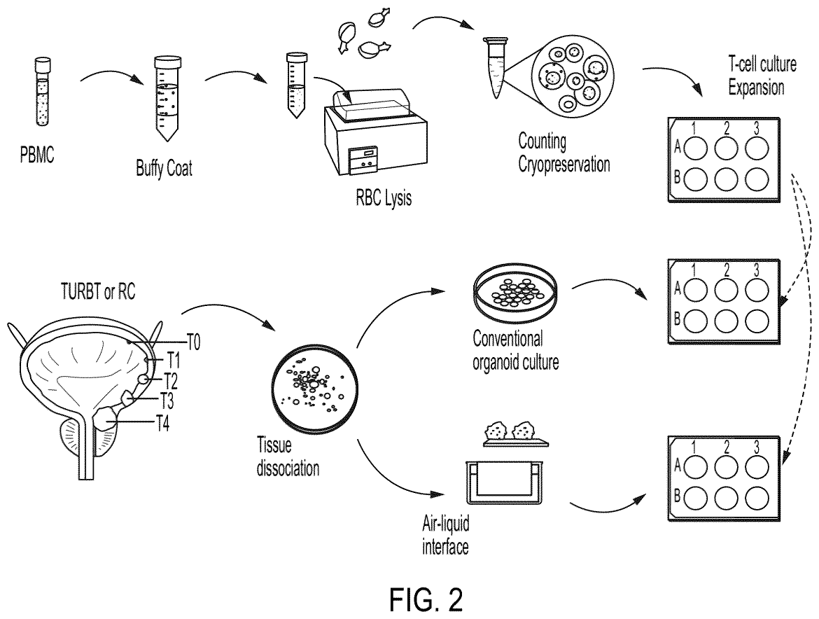

In some embodiments, the tumors are grown in a matrix-like environment. In some embodiments, the tumors are grown in the matrix-like environment of the conventional organoid culture shown in FIG. 2. In some embodiments, the tumors are grown in MATRIGEL®. In some embodiments, the tumors are grown in an air-liquid interphase environment. In some embodiments, the tumors are grown in the air-liquid interphase environment shown in FIG. 2.

In some embodiments, the subject matter described herein relates to immune cells expansion/amplification in vitro. In some embodiments, the immune cells are T lymphocytes. In some embodiments, the T lymphocytes are TILs. In some embodiments, the T lymphocytes are circulating T lymphocytes. In some embodiments, the immune cells expansion is achieved with Interleukin 2 (IL2) treatment. IL2 belongs to a group of related cytokine signaling molecule produced and secreted primarily by leukocytes. IL2 is made by T lymphocytes (T cells) and it increases the growth and activity of other T lymphocytes (T cells) as well as B lymphocytes (B cells). IL2 plays an important role in the development of the immune system. In some embodiments, the amplification is achieved with treatment with a combination of IL2 and beads. In some embodiments, the beads express markers that mimic antigen-presentation. In some embodiments, the immune cells are activated and amplified with treatment with CD3 and/or CD28. In some embodiments, the immune cells are activated and amplified with DYNABEADS™ Human T-Activator CD3/CD28. In some embodiments, the CD3/CD28 are administered concomitantly with IL2 treatment.

In some embodiments, the subject matter described herein relates to activation of peripheral lymphocytes within PBMCs. In some embodiments, the peripheral lymphocytes comprise T cells. In some embodiments, the peripheral T cells are activated with IL2 treatment. In some embodiments, the peripheral T cells are activated with CD3/CD28 treatment. In some embodiments, the peripheral T cells are activated with DYNABEADS™ Human T-Activator CD3/CD28 treatment. In some embodiments, the CD3/CD28 are administered concomitantly with IL2 treatment. In some embodiments, the PMBCs including the peripheral lymphocytes within the PBMCs are cultured. In some embodiments, the PMBCs including the peripheral lymphocytes within PBMCs are co-cultured with organoids. In some embodiments, the organoids are tumor organoids.

In some embodiments, the subject matter described herein relates a method for producing an organoid-immune cells co-culture, the method comprising: a) obtaining a cancer tissue sample from a subject with cancer; b) dissociating the sample of tissue; c) contacting the dissociated tissue with a Matrigel solution and plating in a cell culture support, wherein the Matrigel solution comprises cell culture medium and Matrigel and wherein the Matrigel solution forms a matrix; d) incubating the culture of (c) wherein the dissociated tissue forms organoid-immune cells co-cultures. In some embodiments, the cancer is a bladder cancer, a breast cancer, a prostate cancer, a pancreatic cancer, a kidney tumor, a renal cell carcinoma, a lung tumor, a colorectal cancer, uterine cancer, a thyroid tumor, or a brain cancer. In some embodiments, the cancer is bladder cancer, prostate cancer, or renal cell carcinoma.

In some embodiments, the immune cells comprise T lymphocytes. In some embodiments, the T lymphocytes are tumor-infiltrating lymphocytes (TILs). In some embodiments, the TILs are amplified. In some embodiments, the TILs are amplified with administration of Interleukin 2 (IL2). In some embodiments, the TILs are activated. In some embodiments, the TILs are activated with administration of CD3/CD28 beads. In some embodiments, the amplified and activated TILs are isolated from the co-culture.

In some embodiments, the method comprises less than 40 pipetting steps. In some embodiments, the method comprises two or less centrifugation steps.

In some embodiments, the subject matter described herein relates to using the organoid immune cell co-culture described herein the identify potent TILs capable of killing tumor cells. In some embodiments, the potent TILs are isolated. In some embodiments, the T cell receptors expressed on the surface of the potent TILs are sequenced. In some embodiments, the T cell receptor sequences are cloned into expression vectors. In some embodiments, a library of vectors encoding T cell receptors from potent TILs is generated. In some embodiments, a vector encoding a T cell receptor is introduced in a population of peripheral T cells. In some embodiments, the peripheral T cells are derived from the same subject as the organoid immune cell co-culture used to identify the potent TILs. In some embodiments, the peripheral T cells are naïve T cells. In some embodiments, the peripheral T cells carrying the vector, express the T cell receptor encoded by the vector. In some embodiments, the T cell cells expressing the receptor are amplified or they are amplified and activated. In some embodiments, the amplified T cells are introduced back into the subject they were derived from. In some embodiments, the T cell receptor sequences identify tumor neoantigens. In some embodiments, T cell receptors targeting these neoantigens can be introduced into naïve T cells of a subject with a type of cancer similar to the subject from which the T cell receptor sequence was derived.

The culture conditions of the co-culture described herein can include EGF, 5% fetal bovine serum, and 5% Matrigel. Matrigel™ is the trade name for a reconstituted basement membrane preparation that is extracted from the Engelbreth-Holm-Swarm (EHS) mouse sarcoma, a tumor rich in extracellular matrix proteins. This material, once isolated, is approximately 60% laminin, 30% collagen IV, and 8% entactin. Entactin is a bridging molecule that interacts with laminin and collagen IV, and contributes to the structural organization of these extracellular matrix molecules. Matrigel also contains heparan sulfate proteoglycan (perlecan), TGF-β, epidermal growth factor, insulin like growth factor, fibroblast growth factor, tissue plasminogen activator, and other growth factors which occur naturally in the EHS tumor. There is also residual matrix metalloproteinases derived from the tumor cells. Matrigel is produced and sold by Corning Life Sciences. Trevigen, Inc. markets their own version under the trade name Cultrex® BME.

In some embodiments, organoids of the invention can be cultured in a Matrigel™ gel or matrix. In another embodiment, the organoids of the invention can be co-cultured in a collagen matrix. In one embodiment, the co-culture medium comprises EGF. In another embodiment, the co-culture medium does not comprise EGF. In one embodiment, the co-culture medium comprises GlutaMAX™. In another embodiment, the co-culture medium does not comprise GlutaMAX™. In one embodiment, the co-culture medium comprises antibiotic-antimycotic. In another embodiment, the co-culture medium does not comprise antibiotic-antimycotic.

In one embodiment, the culture medium comprises serum, including, but not limited to, FBS. In another embodiment, the co-culture medium does not comprise serum, including, but not limited to, FBS. In one embodiment, the co-culture medium comprises a ROCK inhibitor. In another embodiment, the co-culture medium does not comprise a ROCK inhibitor. In one embodiment, the co-culture medium comprises Matrigel™. In another embodiment, the co-culture medium does not comprise Matrigel™.

In one aspect, the invention provides a method for culturing a organoids, the method comprising: (a) obtaining a sample of tissue from a subject; (b) dissociating the sample of tissue; (c) contacting the dissociated tissue with a Matrigel™ solution and plating in a cell culture support, wherein the Matrigel™ solution comprises hepatocyte medium and Matrigel™ and wherein the Matrigel™ solution forms a matrix; (d) providing an overlay layer of liquid culture medium comprising hepatocyte medium and FBS; and (e) incubating the culture of (d) wherein the dissociated tissue forms organoids.

In one embodiment, the tissue is non-cancerous. In another embodiment, the tissue is cancerous. In another embodiment, the tissue is obtained from a tumor. In a further embodiment, the subject is a human. In another embodiment, the tissue is obtained from an endoscopic biopsy, an endoscopic resection, or a cystectomy. In a further embodiment, the organoid displays the transformed phenotype of the cancerous tissue. In one embodiment, the culture medium further comprises GlutaMAX™. In another embodiment, the culture medium further comprises EGF. In a further embodiment, the culture medium further comprises antibiotic-antimycotic. In another embodiment, the culture medium comprises 10 ng/ml of EGF. In another embodiment, the culture medium comprises 5% heat-inactivated charcoal stripped FBS. In another embodiment, the culture medium contains a ROCK inhibitor. In another embodiment, the ROCK inhibitor is Y-27632. In another embodiment, the culture medium comprises 10 μM of Y-27632. In some embodiments, the sample of tissue is dissociated with collagenase, hyaluronidase, dispase, or a combination thereof. In some embodiments, the sample of tissue is dissociated with collagenase and hyaluronidase. In some embodiments, the sample of tissue is dissociated with trypsin. In some embodiments, the sample of tissue is dissociated with TrypLE™. In some embodiments, the sample of tissue is dissociated with collagenase and hyaluronidase followed by trypsin. In some embodiments, the sample of tissue is dissociated with collagenase and hyaluronidase followed by TrypLE™. In some embodiments, the method of organoid culturing further comprises serially passaging the organoids. In one embodiment, the organoids are passaged using dispase.

In some embodiment, the contacting of the tissue with Matrigel™ is performed below about 10° C. in order to maintain the Matrigel™ solution in liquid form. After plating in the cell culture support the temperature can be raised above about 10° C. and the Matrigel™ solution can form a matrix or gel. In one embodiment, the Matrigel™ solution solidifies or forms a gel by incubation at 37° C. for 30 minutes. In one embodiment, the Matrigel™ solution solidifies or forms a gel at about 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C.

In another embodiment, before plating the dissociated tissue and Matrigel™ solution in the cell culture support, the cell culture support is surface modified. In one embodiment, the support surface is pre-coated by rinsing Matrigel™ solution over the support surface and incubating the cell culture support at 37° C. for at least 30 minutes. In one embodiment, the Matrigel™ solution comprises hepatocyte medium and Matrigel™. In one embodiment, the Matrigel™ solution comprises serum, including, but not limited to, FBS. In another embodiment, the Matrigel solution does not comprise serum, including, but not limited to, FBS. In one embodiment, the Matrigel™ solution comprises 3 parts Matrigel to 2 parts hepatocyte medium. In one embodiment, the Matrigel™ solution comprises 60% Matrigel™ and 40% hepatocyte medium.

In one aspect, the invention provides a method for culturing a organoid, the method comprising: (a) obtaining a sample of tissue from a subject; (b) dissociating the sample of tissue; (c) contacting the dissociated tissue with a collagen solution and plating in a cell culture support, wherein the collagen solution forms a matrix; (d) providing an overlay layer of liquid culture medium comprising hepatocyte medium and FBS; and (c) incubating the culture of (d) wherein the dissociated tissue forms organoids.

In one embodiment, a sample of tissue can be obtained by biopsy. Methods of obtaining tissue samples are known to one of skill in the art. In one embodiment, the sample of tissue is obtained from a biopsy or endoscopic resection. In another embodiment, the sample of tissue is obtained from a cystectomy.

In one embodiment, the subject is an animal. In other embodiments, the subject is a human. In other embodiments, the subject is a mammal. In some embodiments, the subject is a rodent, such as a mouse or a rat. In some embodiments, the subject is a cow, pig, sheep, goat, cat, horse, dog, and/or any other species of animal used as livestock or kept as pets.

In one aspect, the invention provides a method for culturing a organoid, wherein the organoid maintains or displays the phenotype of the sample of tissue from which the organoid is derived. The phenotype of the organoid can be determined by evaluating markers. Expression of markers can be evaluated by a variety of methods known in the art. In one embodiment, the organoids display the differentiation of the non-cancerous tissue. In one embodiment, the organoids display the transformed phenotype of the cancerous tissue.

In one embodiment, dissociated tissue is separated from the dissociating medium by centrifugation. In one embodiment, the tissue can be further dissociated by incubation of the tissue with TrypLE™. TrypLE™ is an animal origin-free recombinant enzyme alternative to porcine or bovine trypsin. TrypLE™ cleaves peptide bonds on the C-terminal side of lysine and arginine. In one embodiment, the tissue can be further dissociated by incubation of the tissue with trypsin. In one embodiment, the sample is incubated for 3 minutes at 37° C. In one embodiment, the sample is incubated for at least 1 minute, 2 minutes, 3 minutes, 4 minutes, or 5 minutes. In one embodiment, the sample is incubated at about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C. In one embodiment, TrypLE™ or trypsin activity is stopped by the addition of HBSS containing 2% FBS. In one embodiment, the HBSS does not contain Ca²⁺. In another embodiment, the HBSS does not contain Mg²⁺. In one embodiment, the HBSS contains Ca²⁺. In another embodiment, the HBSS contains Mg²⁺. In a further embodiment, the HBSS contains 10 mM HEPES. In one embodiment, the HBSS does not contain phenol red. In another embodiment, the HBSS does contain phenol red. In one embodiment, the HBSS contains at least 0.1% FBS, at least 0.2% FBS, at least 0.3% FBS, at least 0.4% FBS, at least 0.5% FBS, at least 0.6% FBS, at least 0.7% FBS, at least 0.8% FBS, at least 0.9% FBS, at least 1% FBS, at least 2% FBS, at least 3% FBS, at least 4% FBS, at least 5% FBS, at least 6% FBS, at least 7% FBS, at least 8% FBS, at least 9% FBS, at least 10% FBS, or at least 20% FBS.

In one embodiment, dissociated tissue, for example, dissociated tissue, is separated from the TrypLE™, or trypsin solution by centrifugation.

Various culturing parameters can be used with respect to the organoid being cultured. Appropriate culture conditions for organoids can be determined by the skilled artisan. Commercially available medium can be utilized. Non-limiting examples of medium include, for example, Dulbecco's Modified Eagle Medium (DMEM, Life Technologies), Dulbecco's Modified Eagle Medium/Nutrient Mixture F-12 (DMEM/F-12, Life Technologies), Minimal Essential Medium (MEM, Sigma, St. Louis, MO), and hepatocyte medium.

The media described above can be supplemented as necessary with supplementary components or ingredients, including optional components, in appropriate concentrations or amounts, as necessary or desired. Organoid medium solutions provide at least one component from one or more of the following categories: (1) an energy source, usually in the form of a carbohydrate such as glucose; (2) all essential amino acids, and usually the basic set of twenty amino acids plus cysteine; (3) vitamins and/or other organic compounds required at low concentrations; (4) free fatty acids or lipids, for example linoleic acid; and (5) trace elements, where trace elements are defined as inorganic compounds or naturally occurring elements that are typically required at very low concentrations, usually in the micromolar range. The medium also can be supplemented electively with one or more components from any of the following categories: (1) salts, for example, magnesium, calcium, and phosphate; (2) hormones and other growth factors such as, serum, insulin, transferrin, epidermal growth factor and fibroblast growth factor; (3) protein and tissue hydrolysates, for example peptone or peptone mixtures which can be obtained from purified gelatin, plant material, or animal byproducts; (4) nucleosides and bases such as, adenosine, thymidine, and hypoxanthine; (5) buffers, such as HEPES; (6) antibiotics, such as gentamycin or ampicillin; (7) cell protective agents, for example, pluronic polyol; and (8) galactose. The mammalian organoid culture that can be used with the present invention is prepared in a medium suitable for the particular organoid being cultured. In one embodiment, the culture medium can be one of the aforementioned (for example, DMEM, or basal hepatocyte medium) that is supplemented with serum from a mammalian source (for example, fetal bovine scrum (FBS)). For example, Hepatocyte Medium supplemented with FBS can be used to sustain the growth of epithelial organoids. In another embodiment, the medium can be DMEM.

In some embodiments, the methods described herein further comprise evaluating cellular heterogeneity of the organoid-immune cells co-culture. In some embodiments, the methods described herein further comprising evaluating cellular heterogeneity of the cancer tissue sample. In some embodiments, the methods described herein further comprise evaluating cellular heterogeneity of the organoid-immune cells co-culture and the cancer tissue sample. Cellular heterogeneity can be evaluated using techniques known in the art, such as, but not limited to, sequencing techniques such as single-cell sequencing or single nuclei sequencing. In some embodiments, the cellular heterogeneity is evaluated using single nuclei sequencing.

In some embodiments, the evaluation of cellular heterogeneity comprises lysing cells of a sample; generating a single nuclei suspension; and performing single nuclei sequencing. In some embodiments, a sample can be a tumor biopsy from the subject (e.g., a cancer tissue sample). In some embodiments, a sample can be an organoid-immune cell co-culture. In some embodiments, the sequencing results can be annotated using a reference dataset to identify cells within the sample. In some embodiments, the sequencing results can be annotated to identify immune cells within the sample. In some embodiments, the identified cell types can be used to design new studies using the organoid-immune cells co-culture described herein. In some embodiments, lysing cells of a sample comprises lysing the sample on ice in lysis buffer. In some embodiments, the sample is a frozen sample. In some embodiments, lysis is stopped by diluting the lysed sample in buffer (e.g. PBS). In some embodiments, the single nuclei suspension is generated by precipitating and filtering the lysed sample. In some embodiments, the nuclei quality of the single nuclei suspension is evaluated by evaluating nuclear morphology. In some embodiments, the method of evaluating cellular heterogeneity is continued only for samples wherein the single nuclei suspension comprises nuclei of high quality. In some embodiments, nuclei of the single nuclei suspension are tagged with an antibody. In some embodiments, nuclei of the single nuclei suspension are tagged with an anti-nucleoporin antibody.

In some embodiments, the evaluation of cellular heterogeneity can be performed for two or more samples simultaneously. In such an embodiments, evaluation of cellular heterogeneity comprises lysing cells of each sample; generating a single nuclei suspension for each sample; tagging nuclei of the single cell suspension for each sample with a unique antibody; pooling the single nuclei suspensions of all samples; and performing single nuclei sequencing. In some embodiments, a sample can be a tumor biopsy from the subject (e.g., a cancer tissue sample). In some embodiments, a sample can be an organoid-immune cell co-culture. In some embodiments, the sequencing results can be annotated using a reference dataset to identify cells within the sample. In some embodiments, the sequencing results can be annotated to identify immune cells within the sample. In some embodiments, the identified targets can be used to design new studies using the organoid-immune cells co-culture described herein. In some embodiments, lysing cells of a sample comprises lysing each sample on ice in lysis buffer. In some embodiments, the sample is a frozen sample. In some embodiments, lysis of each sample is stopped by diluting the lysed sample in buffer (e.g. PBS). In some embodiments, the single nuclei suspension is generated by precipitating and filtering the lysed sample. In some embodiments, the nuclei quality of the single nuclei suspension for each sample is evaluated by evaluating nuclear morphology. In some embodiments, the method of evaluating cellular heterogeneity is continued only for samples wherein the single nuclei suspension comprises nuclei of high quality. In some embodiments, the nuclei of the single nuclei suspension for each sample are tagged with a unique anti-nucleoporin antibody. In some embodiments, the single nuclei suspensions are pooled and counted.

Method for Identifying a Compound for Administration to a Subject with Cancer

In certain aspects, the subject matter described herein provides a method for identifying a compound for administration to a subject with cancer, the method comprising: a) producing an organoid-immune cells co-culture from a cancer tissue sample from a subject with cancer; b) contacting the organoid-immune cells co-culture with a test compound; c) determining whether growth of the organoid is inhibited in the presence of the test compound, as compared to growth of the organoid in the absence of the test compound; wherein inhibition of growth of the organoid indicates identification of the test compound for administration to said subject.

In some embodiments, the method further comprises adding cell culture media to the organoid-immune cells co-culture prior to the contacting of b), wherein the media comprises Interleukin 2 (IL2). In some embodiments, the method further comprises adding CD3/CD28 beads to the organoid-immune cells co-culture prior to the contacting of b). In some embodiments, the cell culture media comprising IL2 further comprises CD3/CD28 beads.

In certain aspects, the subject matter described herein provides a method for treating cancer in a subject in need thereof, comprising: a) obtaining a cancer tissue sample from a subject with cancer; b) dissociating the sample of tissue; c) contacting the dissociated tissue with a Matrigel™ solution and plating in a cell culture support, wherein the Matrigel solution comprises cell culture medium and Matrigel™ and wherein the Matrigel™ solution forms a matrix; d) incubating the culture of (c), wherein the dissociated tissue forms organoid-immune cells co-culture; c) contacting the organoid-immune cells co-culture with a test compound; and f) determining whether growth of the organoid is inhibited in the presence of the test compound, as compared to growth of the organoid in the absence of the test compound, wherein the test compound is administered to the subject if growth of the organoid is inhibited in the presence of the test compound.

In some embodiments, the method further comprises adding cell culture media to the organoid-immune cells co-culture prior to the contacting of e), wherein the media comprises Interleukin 2 (IL2). In some embodiments, the method further comprises adding CD3/CD28 beads to the organoid-immune cells co-culture prior to the contacting of e). In some embodiments, the cell culture media comprising IL2 further comprises CD3/CD28 beads.

In some embodiments, the cancer is a bladder cancer, a breast cancer, a prostate cancer, a pancreatic cancer, a kidney tumor, a renal cell carcinoma, a lung tumor, a colorectal cancer, uterine cancer, a thyroid tumor, or a brain cancer. In some embodiments, the cancer is bladder cancer, prostate cancer, or renal cell carcinoma. In some embodiments, the subject has an autoimmune disorder. In some embodiments, the subject is at high risk of immune adverse events. In some embodiments, the test compound is an immunotherapy. In some embodiments, the test compound is Pembrolizumab, Nivolumab, Nivo/Ipilimumab, Durvalumab, Avelumab, Pembro/Axitinib, Nivolumab/Cabozantinib, Enfortumab Vedotin, or any combination thereof. In some embodiments, the test compound is administered to the subject if it is determined to inhibit growth of the organoid. In some embodiments, the test compound is not administered to the subject if it is determined not to inhibit growth of the organoid.

In some embodiments, the co-culture described herein can be used to test various therapies or immunotherapies. In some embodiments, the organoid-immune cells co-culture described herein can be used to test for therapy efficacy before the therapy is administered to the patient from whom the organoid-immune cells co-culture is derived. In some embodiments, the immunotherapy tested is anti-PD1 inhibitors (e.g., Pembrolizumab). In some embodiments, the testing of therapies includes contacting the drug of interest used in the tested therapy with the organoid-immune cells co-culture described herein. In some embodiments, the drug is incubated for a period of time with the co-culture. In some embodiments, several doses of the drug are sequentially applied to the co-culture. In some embodiments, various dosages of the drug are applied to the co-culture. In some embodiments, drug efficacy is determined by cell count of surviving tumor cells. In some embodiments, drug efficacy is determined by staining for dead or dying tumor cells. In some embodiments, drug efficacy is determined by staining for surviving tumor cells. In some embodiment, this testing prior to patient administration allows for only effective therapies to be administered to patients, decreasing side effects and time wasted on treatment with ineffective methods.

In some embodiments, the co-culture described herein can be used for testing of immunotherapy agents that would give patients the best response. New and emerging immunotherapy approaches are being routinely tested in early-phase clinical trials, often based on their efficacy in animal models of cancer. In some embodiments, the subject matter described herein relates to an organoid-immune cells co-culture that can improve the process of immunotherapy development by improving pre-clinical testing. This approach is also more cost-effective than clinical trials. In a subset of tumors, there are amplified TILs without tumor killing ability. This suggests that the TILs are present but inhibitory mechanisms prevent them from being activated or targeting the tumors efficiently. These tumors, in particular, would be useful for testing new immunotherapies aiming to release the inhibitory mechanisms and to activate TILs.

In some embodiments, the subject matter described herein relates to screening for immunotherapy sensitivity. Small biopsies from tumors can be taken at patient diagnosis for organoid-immune cells co-culture to test against a variety of available immunotherapies and their various combinations. These immunotherapies include but are not limited to Pembrolizumab, Nivolumab, Nivo/Ipilimumab, Durvalumab, Avelumab, Pembro/Axitinib, Nivolumab/Cabozantinib, Enfortumab Vedotin, etc. Based on the effect of these agents on tumor organoid killing or proliferation, specific recommendations can be made for each patient from whom the tumor organoid was derived. Current predictive methods include CPS (combined proportional score), TPS (tumor proportional score), and TMB (tumor mutational burden), but none of these methods are direct predictors of efficacy. There are patients with high CPS/TMB with no response to immunotherapy and patients with low CPS/TMB with remarkable response to immunotherapy, suggesting that multiple factors are affecting the immune system's ability to specifically target cancer cells. The co-culture described herein allows for the native tumor to be tested ex vivo with specific agents for their ability to stimulate killing. Given the minimal manipulation involved, the testing method described herein can also be offered to specific subsets of patients where other methods are prohibitive or have already failed, such as patients at high risk for immune adverse effects or patients for whom no other options are available.

In some embodiments, the organoid-immune cells co-culture described herein can be used to predict patient response to existing and emerging therapies. In some embodiments, a phase I/II clinical trial platform can include the organoid-immune cells co-cultures described herein at the time of trial enrollment for drug pre-testing. In some embodiments, the organoid-immune cells co-cultures described herein can be utilized in parallel testing of therapeutic drugs in the organoid-immune cells co-culture and in patients receiving the same drugs. In some embodiments, the organoid-immune cells co-culture described herein can be utilized in correlation study between patient tumor response and organoid response to therapy. In some embodiments, the organoid-immune cells co-culture described herein can be utilized in evaluating the mechanisms of therapy resistance in co-clinical studies, where the preclinical studies and clinical trials are conducted simultaneously. In some embodiments, the organoid-immune cells co-culture described herein can be utilized in addition to performing a corresponding single-cell sequencing and computational analysis to evaluate the characteristics of tumors that respond compared to those that do not respond to treatment. In some embodiments, the organoid-immune cells co-culture described herein can be utilized with multiplex immunostaining to evaluate the tumor microenvironment and identify mechanisms of drug resistance. In some embodiments, identified targets can be used to design new studies using the organoid-immune cells co-culture described herein, possibly with expansion to incorporate more of the microenvironment components into the co-culture described herein. In some embodiments, the co-culture described herein can be expanded to include macrophages. In some embodiments, the organoid-immune cells co-culture described herein can be used to design investigator-initiated trials based on identified drug targets.

In some embodiments, the methods described herein further comprise evaluating cellular heterogeneity of the organoid-immune cells co-culture. In some embodiments, the methods described herein further comprising evaluating cellular heterogeneity of the cancer tissue sample. In some embodiments, the methods described herein further comprise evaluating cellular heterogeneity of the organoid-immune cells co-culture and the cancer tissue sample. Cellular heterogeneity can be evaluated using techniques known in the art, such as, but not limited to, sequencing techniques such as single-cell sequencing or single nuclei sequencing. In some embodiments, the cellular heterogeneity is evaluated using single nuclei sequencing.

In some embodiments, the evaluation of cellular heterogeneity comprises lysing cells of a sample; generating a single nuclei suspension; and performing single nuclei sequencing. In some embodiments, a sample can be a tumor biopsy from the subject (e.g., a cancer tissue sample). In some embodiments, a sample can be an organoid-immune cell co-culture. In some embodiments, the sequencing results can be annotated using a reference dataset to identify cells within the sample. In some embodiments, the sequencing results can be annotated to identify immune cells within the sample. In some embodiments, the identified cell types can be used to design new studies using the organoid-immune cells co-culture described herein. In some embodiments, lysing cells of a sample comprises lysing the sample on ice in lysis buffer. In some embodiments, the sample is a frozen sample. In some embodiments, lysis is stopped by diluting the lysed sample in buffer (e.g. PBS). In some embodiments, the single nuclei suspension is generated by precipitating and filtering the lysed sample. In some embodiments, the nuclei quality of the single nuclei suspension is evaluated by evaluating nuclear morphology. In some embodiments, the method of evaluating cellular heterogeneity is continued only for samples wherein the single nuclei suspension comprises nuclei of high quality. In some embodiments, nuclei of the single nuclei suspension are tagged with an antibody. In some embodiments, nuclei of the single nuclei suspension are tagged with an anti-nucleoporin antibody.

In some embodiments, the evaluation of cellular heterogeneity can be performed for two or more samples simultaneously. In such an embodiment, evaluation of cellular heterogeneity comprises lysing cells of each sample; generating a single nuclei suspension for each sample; tagging nuclei of the single cell suspension for each sample with a unique antibody; pooling the single nuclei suspensions of all samples; and performing single nuclei sequencing. In some embodiments, a sample can be a tumor biopsy from the subject (e.g., a cancer tissue sample). In some embodiments, a sample can be an organoid-immune cell co-culture. In some embodiments, the sequencing results can be annotated using a reference dataset to identify cells within the sample. In some embodiments, the sequencing results can be annotated to identify immune cells within the sample. In some embodiments, the identified targets can be used to design new studies using the organoid-immune cells co-culture described herein. In some embodiments, lysing cells of a sample comprises lysing each sample on ice in lysis buffer. In some embodiments, the sample is a frozen sample. In some embodiments, lysis of each sample is stopped by diluting the lysed sample in buffer (e.g. PBS). In some embodiments, the single nuclei suspension is generated by precipitating and filtering the lysed sample. In some embodiments, the nuclei quality of the single nuclei suspension for each sample is evaluated by evaluating nuclear morphology. In some embodiments, the method of evaluating cellular heterogeneity is continued only for samples wherein the single nuclei suspension comprises nuclei of high quality. In some embodiments, the nuclei of the single nuclei suspension for each sample are tagged with a unique anti-nucleoporin antibody. In some embodiments, the single nuclei suspensions are pooled and counted.

Method for Identifying a Compound that Inhibits Cancer

In certain aspects, the subject matter described herein provides a method for identifying a compound that inhibits cancer, the method comprising: a) producing an organoid-immune cells co-culture from a cancer tissue sample from a subject with cancer; b) contacting the organoid-immune cells co-culture with a test compound; c) determining whether growth of the organoid is inhibited in the presence of the test compound, as compared to growth of the organoid in the absence of the test compound; wherein inhibition of growth of the organoid indicates identification of a compound that inhibits cancer.

In some embodiments, the method further comprises adding cell culture media to the organoid-immune cells co-culture prior to the contacting of b), wherein the media comprises Interleukin 2 (IL2). In some embodiments, the method further comprises adding CD3/CD28 beads to the organoid-immune cells co-culture prior to the contacting of b). In some embodiments, the cell culture media comprising IL2 further comprises CD3/CD28 beads.

In certain aspects, the subject matter described here provides a method for identifying a compound that inhibits cancer, the method comprising: a) producing a library of organoid-immune cells co-cultures from cancer tissue samples from a plurality of subjects with cancer; b) contacting the organoid-immune cells co-culture library with a test compound; c) determining whether growth of one or more organoids of the library are inhibited in the presence of the test compound, as compared to growth of that organoid of the library in the absence of the test compound; wherein inhibition of growth of one or more organoids of the library indicates identification of a compound that inhibits cancer.

In some embodiments, the method further comprises adding cell culture media to the organoid-immune cells co-culture library prior to the contacting of b), wherein the media comprises Interleukin 2 (IL2). In some embodiments, the method further comprises adding CD3/CD28 beads to the organoid-immune cells co-culture library prior to the contacting of b).

In some embodiments, the cell culture media comprising IL2 further comprises CD3/CD28 beads.

In some embodiments, inhibition of growth of a significant number of organoids of the library indicates identification of a compound that inhibits cancer. In some embodiments, the cancer is a bladder cancer, a breast cancer, a prostate cancer, a pancreatic cancer, a kidney tumor, a renal cell carcinoma, a lung tumor, a colorectal cancer, uterine cancer, a thyroid tumor, or a brain cancer. In some embodiments, the cancer is bladder cancer, prostate cancer, or renal cell carcinoma.

In some embodiments, the test compound is an antineoplastic agent, a chemotherapy agent, an immunotherapy, or any combination thereof. In some embodiments, the test compound is an immunotherapy. In some embodiments, the test compound is a combination of immunotherapies.

In some embodiments, the methods described herein further comprise evaluating cellular heterogeneity of the organoid-immune cells co-culture. In some embodiments, the methods described herein further comprising evaluating cellular heterogeneity of the cancer tissue sample. In some embodiments, the methods described herein further comprise evaluating cellular heterogeneity of the organoid-immune cells co-culture and the cancer tissue sample. Cellular heterogeneity can be evaluated using techniques known in the art, such as, but not limited to, sequencing techniques such as single-cell sequencing or single nuclei sequencing. In some embodiments, the cellular heterogeneity is evaluated using single nuclei sequencing.

In some embodiments, the evaluation of cellular heterogeneity comprises lysing cells of a sample; generating a single nuclei suspension; and performing single nuclei sequencing. In some embodiments, a sample can be a tumor biopsy from the subject (e.g., a cancer tissue sample). In some embodiments, a sample can be an organoid-immune cell co-culture. In some embodiments, the sequencing results can be annotated using a reference dataset to identify cells within the sample. In some embodiments, the sequencing results can be annotated to identify immune cells within the sample. In some embodiments, the identified cell types can be used to design new studies using the organoid-immune cells co-culture described herein. In some embodiments, lysing cells of a sample comprises lysing the sample on ice in lysis buffer. In some embodiments, the sample is a frozen sample. In some embodiments, lysis is stopped by diluting the lysed sample in buffer (e.g. PBS). In some embodiments, the single nuclei suspension is generated by precipitating and filtering the lysed sample. In some embodiments, the nuclei quality of the single nuclei suspension is evaluated by evaluating nuclear morphology. In some embodiments, the method of evaluating cellular heterogeneity is continued only for samples wherein the single nuclei suspension comprises nuclei of high quality. In some embodiments, nuclei of the single nuclei suspension are tagged with an antibody. In some embodiments, nuclei of the single nuclei suspension are tagged with an anti-nucleoporin antibody.

In some embodiments, the evaluation of cellular heterogeneity can be performed for two or more samples simultaneously. In such an embodiments, evaluation of cellular heterogeneity comprises lysing cells of each sample; generating a single nuclei suspension for each sample; tagging nuclei of the single cell suspension for each sample with a unique antibody; pooling the single nuclei suspensions of all samples; and performing single nuclei sequencing. In some embodiments, a sample can be a tumor biopsy from the subject (e.g., a cancer tissue sample). In some embodiments, a sample can be an organoid-immune cell co-culture. In some embodiments, the sequencing results can be annotated using a reference dataset to identify cells within the sample. In some embodiments, the sequencing results can be annotated to identify immune cells within the sample. In some embodiments, the identified targets can be used to design new studies using the organoid-immune cells co-culture described herein. In some embodiments, lysing cells of a sample comprises lysing each sample on ice in lysis buffer. In some embodiments, the sample is a frozen sample. In some embodiments, lysis of each sample is stopped by diluting the lysed sample in buffer (e.g. PBS). In some embodiments, the single nuclei suspension is generated by precipitating and filtering the lysed sample. In some embodiments, the nuclei quality of the single nuclei suspension for each sample is evaluated by evaluating nuclear morphology. In some embodiments, the method of evaluating cellular heterogeneity is continued only for samples wherein the single nuclei suspension comprises nuclei of high quality. In some embodiments, the nuclei of the single nuclei suspension for each sample are tagged with a unique anti-nucleoporin antibody. In some embodiments, the single nuclei suspensions are pooled and counted.

Populations of T-Lymphocytes and Methods of Treating Cancer with Same

In certain aspects, the subject matter described herein provides a method of identifying tumor-infiltrating lymphocytes (TILs), the method comprising: a) producing an organoid-immune cells co-culture from a cancer tissue sample from a subject with cancer; b) subjecting the organoid-immune cells co-culture to conditions wherein if tumor-infiltrating lymphocytes (TILs) are present within the organoid-immune cells co-culture they are expanded; c) subjecting the organoid-immune cells co-culture to conditions wherein if TILs are present within the organoid-immune cells co-culture they are activated; d) determining whether growth of the organoid is inhibited by the activated TILs, wherein: (i) inhibition of growth of the organoid indicates identification of TILs within the organoid-immune cells co-culture or (ii) lack of inhibition of growth of the organoid indicates absence of TILs within the organoid-immune cells co-culture.

In certain aspects, the subject matter described herein provides a method for treating cancer in a subject in need thereof, comprising: a) producing an organoid-immune cells co-culture from a cancer tissue sample from a subject with cancer; b) subjecting the organoid-immune cells co-culture to conditions wherein if tumor-infiltrating lymphocytes (TILs) are present within the organoid-immune cells co-culture they are expanded; c) subjecting the organoid-immune cells co-culture to conditions wherein if TILs are present within the organoid-immune cells co-culture they are activated; determining whether growth of the organoid is inhibited by the activated TILs, wherein: (i) inhibition of growth of the organoid indicates identification of TILs within the organoid-immune cells co-culture and a therapeutically effective amount of the said TILs are administered to the subject, or (ii) lack of inhibition of growth of the organoid indicates absence of TILs within the organoid and a therapeutically effective amount of TILs are not administered to the subject.

In some embodiments, steps b) and c) are performed concurrently. In some embodiments, if TILs are identified within the organoid-immune cells co-culture, the method further comprises isolating or separating TILs from the organoid-immune cells co-culture. In some embodiments, the isolated or separated TILs are expanded by culturing the cells in a cell culture medium. In some embodiments, the isolated or separated TILs are expanded by culturing the cells in a cell culture medium with allogenic irradiated PBMC feeder cells. In some embodiments, the cell culture medium is a non-Matrigel™ containing T-cell medium. In some embodiments, the isolated or separated TILs are expanded by culturing the cells for seven days in a mixture of TIL-CM and AIM-V medias. In some embodiments, the wherein the isolated or separated TILs are further expanded by culturing the cells for an additional seven days in AIM-V medium.

In some embodiments, if TILs are identified within the organoid-immune cells co-culture, the method further comprises isolating or separating TILs from the organoid-immune cells co-culture and expanding the isolated or separated TILs by culturing the cells in a cell culture medium before said TILs are administered to the subject.

In some embodiments, step b) comprises adding cell culture media comprising Interleukin 2 (IL2) to the organoid-immune cells co-culture. In some embodiments, step c) comprises adding CD3/CD28 beads to the organoid-immune cells co-culture. In some embodiments, the administration comprises intravenous administration of the TILs. In some embodiments, if there is a lack of inhibition of growth of the organoid indicating absence of TILs within the organoid-immune cells co-culture, the method further comprises: c) adding a cell culture media comprising PBMCs, IL2, and CD3/CD28 beads to the organoid-immune cells co-culture; f) determining whether growth of the organoid is inhibited, wherein: (i) inhibition of growth of the organoid indicates identification of peripheral lymphocytes within the PBMCs that can target the organoid or (ii) lack of inhibition of growth of the organoid indicates absence of peripheral lymphocytes within the PBMCs that can target the organoid. In some embodiments, the PBMCs are obtained from the subject. In some embodiments, if peripheral lymphocytes that can target the organoid are identified within the PBMCs, the method further comprises isolating or separating said peripheral lymphocytes from the organoid-immune cells co-culture. In some embodiments, the isolated or separated peripheral lymphocytes are expanded by culturing the cells in a cell culture medium. In some embodiments, the isolated or separated peripheral lymphocytes are expanded by culturing the cells in a cell culture medium with allogenic irradiated PBMC feeder cells.

In some embodiments, the cell culture medium is a non-Matrigel™ containing T-cell medium. In some embodiments, the isolated or separated peripheral lymphocytes are expanded by culturing the cells for seven days in a mixture of TIL-CM and AIM-V medias. In some embodiments, the wherein the isolated or separated peripheral lymphocytes are further expanded by culturing the cells for an additional seven days in AIM-V medium.

In some embodiments, if peripheral lymphocytes are identified that can target the organoid are identified within the PBMCs, the method further comprises isolating or separating said peripheral lymphocytes from the organoid-immune cells co-culture and expanding the isolated or separated peripheral lymphocytes by culturing the cells in a cell culture medium and administering said peripheral lymphocytes to the subject. In some embodiments, step b) comprises adding cell culture media comprising Interleukin 2 (IL2) to the organoid-immune cells co-culture. In some embodiments, step c) comprises adding CD3/CD28 beads to the organoid-immune cells co-culture. In some embodiments, the administration comprises intravenous administration of the peripheral lymphocytes.

In some embodiments, the cancer is a bladder cancer, a breast cancer, a prostate cancer, a pancreatic cancer, a kidney tumor, a renal cell carcinoma, a lung tumor, a colorectal cancer, uterine cancer, a thyroid tumor, or a brain cancer. In some embodiments, the cancer is bladder cancer, prostate cancer, or renal cell carcinoma.

In some embodiments, if TILs are identified within the organoid-immune cells co-culture, the method further comprises sequencing the nucleic acid sequence encoding one or more T cell receptors on the surface of said TILs.

In some embodiments, if peripheral lymphocytes that can target the organoid are identified within the PBMCs the method further comprises sequencing the nucleic acid sequence encoding one or more T cell receptors on the surface of said peripheral lymphocytes. In some embodiments, the nucleic acid sequence of a T cell receptor is inserted in a vector. In some embodiments, the vector is introduced into peripheral T cells isolated from the subject's PBMCs.

In some embodiments, the peripheral T cells express the T cell receptor encoded by the nucleic acid sequence. In some embodiments, the peripheral T cells expressing the T cell receptor are selected for their ability to kill organoid cancer cells. In some embodiments, the selected T cells are expanded. In some embodiments, the expanded T cells are introduced back into the subject.

In certain aspects, the subject matter described herein provides a pharmaceutical composition comprising a therapeutically effective amount of the TILs according to any of the methods described herein.

In certain aspects, the subject matter described herein provides a pharmaceutical composition comprising a therapeutically effective amount of the peripheral lymphocytes according to any of the methods described herein.

T-cell transfer therapy, also called adoptive cell therapy, adoptive immunotherapy, or immune cell therapy, is a type of immunotherapy where a patient's own immune cells attack tumor cells and/or cancer tissues. There are two main types of T-cell transfer therapy: tumor-infiltrating lymphocytes (or TIL) therapy and CAR T-cell therapy. They both involve collecting immune cells from a patient, growing large numbers of these cells ex vivo, and then re-introducing the cells back to the same patient (autologous re-introduction). T-cell therapy for cancer is a promising but costly approach to treating cancer. For example, the estimated CAR-T therapy which includes harvesting, amplification, genetic engineering, and reintroduction can cost upwards of $300K per patient. These T-cells are derived from peripheral blood and therefore might have not seen any tumors. In some embodiments, the subject matter described herein relates to systematically isolating and amplifying tumor-specific infiltrating lymphocytes from a very small tumor sample (˜3 mm tumor). These T-cells can specifically kill the organoids with which they co-exist but not the adjacent tissues. The benefit to this co-culture over an engineered co-culture is as follows: (1) reduced cost by eliminating the genetic engineering step, (2) TILs are tumor specific and therefore have already been primed to kill tumor cells. The advantage of the organoid-immune cells co-culture described herein is the ability to test the T-cells' ability to kill organoids prior to introducing them back into the patient. In some embodiments, the co-culture described herein can be used for optimization of T-cells prior to re-introduction into patients as a form of T-cell therapy. If the amplified TILs are unable to kill organoids, then they might not work in the patient. Excluding these TILs from re-introduction into the patient spares the patient an ineffective treatment, adverse events, and costs associated with it. Currently, no cell-based therapy is tested for efficacy or optimized prior to re-introduction into the patient Thus, there are instances where the patients have adverse events without real clinical benefit. The advantage of the co-culture to test efficacy and select for tumor-killing ability before the therapies are given to the patient is particularly important for T-cell therapies such as TILs therapy or CAR-T therapy because they are known to cause severe adverse effects such as cytokine release syndrome. The ability to select for tumor-specific T-cells means that we can potentially increase effectiveness of cell-based therapy while reducing adverse events.

In some embodiments, therapy with TILs takes advantage of the T cells that are found in the patient's tumor. Prior to treatment, these lymphocytes can be tested ex vivo to determine which ones best recognize the patient's tumor cells. Then, the selected lymphocytes can be treated with substances to grow them in large numbers quickly or to amply them. The advantage of using TILs in cancer therapy is that the patient's own TILs are in or near the tumor and, thus, have already shown the ability to recognize the tumor cells. However, there may not be enough of them available to kill the tumor or to overcome the signals that the tumor is releasing to suppress the immune system. That is why TILs amplification and re-introduction are beneficial in this therapy.

In some embodiments, the subject matter described herein relates to activating lymphocytes to specifically target and/or kill patient-derived tumor organoids. In some embodiments, the normal tumor-adjacent tissues are spared. In some embodiments, the organoid-immune cells co-culture described herein can be used to activate a patient's own lymphocytes to specifically target the patient's tumor cells. In some embodiments, patient-derived lymphocytes can be activated ex vivo. In some embodiments, patient-derived lymphocytes can be amplified ex vivo. In some embodiments, the amplification is with IL2 treatment of the lymphocytes. In some embodiments, the activation and amplification is achieved with CD3/CD28 treatment of the lymphocytes. In some embodiments, the CD3/CD28 treatment is concurrent with the IL2 treatment. In some embodiments, the activated and/or amplified lymphocytes can be re-introduced back into to the patient. In some embodiments, the activated lymphocytes can be utilized in adoptive cell therapy. The T cell therapy can be combined with chemotherapy, radiation therapy, or any suitable cancer treatment known in the art. The treatments can be administered concurrently or consecutively.

In some embodiments, the subject matter described herein relates to generating T cells from peripheral lymphocytes. In some embodiments, the T cells are generated from peripheral lymphocytes of cancer patients whose organoid-immune cells co-culture is immuno poor. In some embodiments, an immuno poor co-culture lacks TILs. In some embodiments, the lack of TILs can be remedied by addition of PBMCs, from the same patient from which the organoid was derived, into the co-culture. In some embodiments, peripherally circulating T cells present in the PBMCs can be expanded and activated in the presence of the organoid. In some embodiments, the T cells can be expanded and activated with addition of IL2 and CD3/CD28 beads to the culture media. In some embodiments, the activated T cells can be trained to kill the tumor cells of the organoid. In some embodiments, these tumor-specific T cells can be introduced back into the same patient from which the PBMCs and the organoid co-culture were derived.

In some embodiments, the organoid-immune cells co-cultures described herein can be generated from repeated tumor biopsies of the same tumor. In some embodiments, these repeated co-cultures recapitulate tumor changes with disease progression. In some embodiments, these repeated co-cultures produce a library of co-cultures recapitulating the evolving tumor. In some embodiments, TILs can be isolated from each of the repeated co-cultures generating a library of TILs specific for the respective tumor stage. In some embodiments, autologous PBMCs can be added to these co-cultures. In some embodiments, the co-cultures, to which PBMCs are added, are immune poor. In some embodiments, peripherally circulating T cells present in the PBMCs can be trained to kill tumor cells of the organoid following expansion and activation with IL2 and CD3/CD28 beads. In some embodiments, these trained T cells can be introduced back into the patient.

In some embodiments, the subject matter described herein relates to the treatment of cancer. In some embodiments, the cancer is a solid tumor. In some embodiments, the cancer is bladder cancer. In some embodiments, the cancer is kidney cancer. In some embodiments, the cancer is renal cell carcinoma (RCC). In some embodiments, the cancer is breast cancer, prostate cancer, pancreatic cancer, lung cancer, colorectal cancer, uterine cancer, thyroid cancer, or glioma. In some embodiments, the cancer is metastatic. In some embodiments, the cancer is recurrent.

In some embodiments, the subject matter described herein relates to patient treatment with TILs. In some embodiments, the TILS are autologous TILs, derived from the same patient they are using to treat. The tumor-infiltrating lymphocytes described herein can be consistently expanded in the organoid-immune cells co-culture described herein. This co-culture not only amplifies tumor-specific T-cells but can also be predictive of whether the T-cells will be able to kill the tumor. In some embodiments, TILs that specifically kill tumor cells in the organoid-immune cells co-culture can be selected. This co-culture represents a specific method for selecting tumor-infiltrating lymphocytes with the expressed purpose of infusing them back into the patient from whom the organoid and TILs were derived.

Human peripheral blood mononuclear cells (PBMCs) are peripheral blood cells controlling the immune response. (Sen, P. et al. Front. Mol. Biosci. 2018. 4:96). PMBCs include T lymphocytes (T cells; ˜70%), B lymphocytes (B cells; ˜15%), monocytes (˜5%), dendritic cells (˜1%) and natural killer (NK) cells (˜10%). The T cell co-receptor (CD3+ expressing T lymphocytes) can be further sub-divided into CD4+ and CD8+ cytotoxic cells, which are present in PBMCs in approximately 2:1 ratio. B lymphocytes are bone marrow derived cells, which express the B cell receptor and bind to specific antigens against which they initiate antibody responses, thus forming the core of the adaptive humoral immune system. The cytotoxic natural killer cells (NK cells) can directly destroy pathogen-infected cells. Additionally, NK cells secrete lymphokines and interact with other immune cells and thus participate in immune responses by means other than direct cytotoxicity.

Lymphocytes, which include T cells and B cells, are constantly circulating through the bloodstream to search and identify abnormal cells, including cancer. As cancers grow, lymphocytes recognize the cancer cells as abnormal and penetrate into the tumor. These lymphocytes are called the tumor-infiltrating lymphocytes (TILs). In some embodiments the subject matter described herein relates to growing and expanding TILs. In some embodiments, the subject matter disclosed herein relates to preparing and/or maintaining TILs in the organoid immune cells co-cultures described herein. In some embodiments, the TILs are derived from the tumor sample cultured in the organoid immune cells co-culture described herein. In some embodiments, the TILs can be grown and/or amplified by treating the organoid immune cells co-culture described herein with IL2. In some embodiments, TILs can be grown from primary tumors cultured in the co-culture described herein. In some embodiments, the primary tumors are from cancer patients. In some embodiments, the TILs described herein can be grown using various approaches. In some embodiments, the TILs disclosed herein can be grown using any suitable approach known in the field. In some embodiments, the TILs can be re-introduced into patients from which they were obtained as a form of adoptive cell transfer. The current standard in the field is that prior to their re-introduction into patients, TILs are neither tested, nor selected for their efficacy and antigen specificity ex vivo. Also, it has not been demonstrated that TILs can co-exist in culture with patient-derived tumor organoids and display functional activities. In some embodiments, the subject matter described herein relates to establishing an organoid-TILs co-culture. In some embodiments, the organoid is a patient derived tumor organoid. In some embodiments, the organoid-TILs co-culture is used to test cancer therapies and predict the patient response to these therapies. In some embodiments, the organoid-TILs co-culture is used to expand the TILs. In some embodiments, the TILs are expanded by treating the co-culture with IL2. In some embodiments, the organoid-TILs co-culture is used to activate the TILs. In some embodiments, the organoid-TILs co-culture is used to select TILs for their efficacy and specificity to target cancer cells. In some embodiments, the organoid-TILs co-culture or the TILs described herein are used in the treatment of cancer. In some embodiments, the cancer is a solid tumor. In some embodiments, the cancer is bladder cancer. In some embodiments, the cancer is kidney cancer. In some embodiments, the cancer is renal cell carcinoma (RCC). In some embodiments, the cancer is breast cancer, prostate cancer, pancreatic cancer, lung cancer, colorectal cancer, uterine cancer, thyroid cancer, or glioma. In some embodiments, the cancer is metastatic. In some embodiments, the cancer is recurrent.

Previously, a culture system was established to grow tumor organoids from primary patient bladder tumor samples. These three-dimensional models recapitulate properties of their corresponding parental tumors. Organoid cultures can be established efficiently from patient biopsies acquired before and after disease recurrence. Notably, organoid lines often retain parental tumor heterogeneity and exhibit a spectrum of genomic changes that are consistent with tumor evolution in culture. Analyses of drug response using bladder tumor organoids show partial correlations with mutational profiles, as well as changes associated with treatment resistance. Patient-derived bladder tumor organoids represent a faithful model system for studying tumor evolution and treatment response in the context of precision cancer medicine. More information on tumor organoids can be found in Lee, S. H. et al., Cell, 2018, 515-28, which is incorporated herein in its entirety. In some embodiments, the subject matter described herein relates to co-culturing of tumor organoids with immune cells. In some embodiments, the immune cells are T lymphocytes. In some embodiments, the immune cells are TILs. In some embodiments, the tumor is bladder tumor. In some embodiments, the tumor is kidney tumor. In some embodiments, the tumor is obtained from renal cell carcinoma (RCC). In some embodiments, the tumor is breast tumor, prostate tumor, pancreatic tumor, lung tumor, colorectal tumor, uterine tumor, thyroid tumor, or tumor obtained from glioma. In some embodiments, the tumor is from metastatic cancer. In some embodiments, the cancer is recurrent.

In some embodiments, the methods described herein further comprise evaluating cellular heterogeneity of the organoid-immune cells co-culture. In some embodiments, the methods described herein further comprising evaluating cellular heterogeneity of the cancer tissue sample. In some embodiments, the methods described herein further comprise evaluating cellular heterogeneity of the organoid-immune cells co-culture and the cancer tissue sample. Cellular heterogeneity can be evaluated using techniques known in the art, such as, but not limited to, sequencing techniques such as single-cell sequencing or single nuclei sequencing. In some embodiments, the cellular heterogeneity is evaluated using single nuclei sequencing.

In some embodiments, if the organoid-immune cells co-culture and/or cancer tissue sample are identified as lacking CD8+ T cells, the method further comprises proceeding with the various methods described herein for administration of the subject's autologous PBMC. For example, the method further comprises: c) adding a cell culture media comprising PBMCs, IL2, and CD3/CD28 beads to the organoid-immune cells co-culture; and f) determining whether growth of the organoid is inhibited, wherein: (i) inhibition of growth of the organoid indicates identification of peripheral lymphocytes within the PBMCs that can target the organoid or (ii) lack of inhibition of growth of the organoid indicates absence of peripheral lymphocytes within the PBMCs that can target the organoid. In some embodiments, the PBMCs are obtained from the subject. In some embodiments, if peripheral lymphocytes that can target the organoid are identified within the PBMCs, the method further comprises isolating or separating said peripheral lymphocytes from the organoid-immune cells co-culture.

In some embodiments, the evaluation of cellular heterogeneity comprises lysing cells of a sample; generating a single nuclei suspension; and performing single nuclei sequencing. In some embodiments, a sample can be a tumor biopsy from the subject (e.g., a cancer tissue sample). In some embodiments, a sample can be an organoid-immune cell co-culture. In some embodiments, the sequencing results can be annotated using a reference dataset to identify cells within the sample. In some embodiments, the sequencing results can be annotated to identify immune cells within the sample. In some embodiments, the identified cell types can be used to design new studies using the organoid-immune cells co-culture described herein. In some embodiments, lysing cells of a sample comprises lysing the sample on ice in lysis buffer. In some embodiments, the sample is a frozen sample. In some embodiments, lysis is stopped by diluting the lysed sample in buffer (e.g. PBS). In some embodiments, the single nuclei suspension is generated by precipitating and filtering the lysed sample. In some embodiments, the nuclei quality of the single nuclei suspension is evaluated by evaluating nuclear morphology. In some embodiments, the method of evaluating cellular heterogeneity is continued only for samples wherein the single nuclei suspension comprises nuclei of high quality. In some embodiments, nuclei of the single nuclei suspension are tagged with an antibody. In some embodiments, nuclei of the single nuclei suspension are tagged with an anti-nucleoporin antibody.

In some embodiments, the evaluation of cellular heterogeneity can be performed for two or more samples simultaneously. In such an embodiment, evaluation of cellular heterogeneity comprises lysing cells of each sample; generating a single nuclei suspension for each sample; tagging nuclei of the single cell suspension for each sample with a unique antibody; pooling the single nuclei suspensions of all samples; and performing single nuclei sequencing. In some embodiments, a sample can be a tumor biopsy from the subject (e.g., a cancer tissue sample). In some embodiments, a sample can be an organoid-immune cell co-culture. In some embodiments, the sequencing results can be annotated using a reference dataset to identify cells within the sample. In some embodiments, the sequencing results can be annotated to identify immune cells within the sample. In some embodiments, the identified targets can be used to design new studies using the organoid-immune cells co-culture described herein. In some embodiments, lysing cells of a sample comprises lysing each sample on ice in lysis buffer. In some embodiments, the sample is a frozen sample. In some embodiments, lysis of each sample is stopped by diluting the lysed sample in buffer (e.g. PBS). In some embodiments, the single nuclei suspension is generated by precipitating and filtering the lysed sample. In some embodiments, the nuclei quality of the single nuclei suspension for each sample is evaluated by evaluating nuclear morphology. In some embodiments, the method of evaluating cellular heterogeneity is continued only for samples wherein the single nuclei suspension comprises nuclei of high quality. In some embodiments, the nuclei of the single nuclei suspension for each sample are tagged with a unique anti-nucleoporin antibody. In some embodiments, the single nuclei suspensions are pooled and counted.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1—Organoid-Immune Cells Co-Culture Protocol

In some embodiments, the subject matter disclosed herein relates to a protocol for establishing of an organoid-immune cells co-culture. In some embodiments, the organoid is a tumor organoid. In some embodiments, the protocol described herein emphasizes physical dissociation of the isolated tumor sample to maximize cell yield. In some embodiments, the tumor sample is isolated from a patient. In some embodiments, the protocol described herein de-emphasizes manipulation by teaching fewer pipetting steps (total 40×) and fewer spin steps (total 2 spins) compared to protocols known in the art. In some embodiments, de-emphasizing manipulation prevents cell senescence and increases cell yield. In some embodiments, the protocol described herein uses a large 300 micron filter to increase yield of small clusters of cells as opposed to only selecting for single cells with 100 micron filters.

### A. Organoid and T-Cell Extraction Procedure

1. Tissues can be prioritized as follows:
   a. Organoids+TILs (tumor-infiltrating lymphocytes)
   b. Frozen tumor (preferred) or block
   c. FFPE (can always requested from pathology later if necessary)
2. If organoids+TILs are used, place a piece of tissue in 300 μl of media in 1.5 ml tube
3. Use spring scissors to mince the tissue sample
4. Place minced tissue back into 50 ml tube with 10 ml of media with 1× hyaluronidase/collagenase
5. Digest tissue with hyaluronidase/collagenase for 10 min at 37° C.
6. Resuspend tissue by pipetting up and down 20 times
7. Filter tissue through a 300-micron filter into a new 50 ml tube (tube A: filtered)
8. Invert filter and move large pieces of tissue back into a 1.5 ml Eppendorf tube
9. Take 5 ml of TrypLE to rinse the inverted filter into a new 50 ml tube (tube B: unfiltered) to move all residual tissue into the TrypLE
10. Use 300 ul of TrypLE and spring scissors to further mince large chunks of tissues in the 1.5 ml Eppendorf. Transfer this mixture into tube B with TrypLE after sufficient dissociation (2 min).
11. Incubate tissues in TrypLE (tube B) at 37° C. for 3 min
12. Pipet digestion mixture (tube B) up and down 20 times
13. Filter the mixture from tube B through the same 300-micron filter into the filtered collagenase/hyaluronidase mixture of cells (tube A)
14. Spin cells at 300×g for 5 min
15. Rinse cells with 5 ml of media, spin cells at 300×g for 5 min
16. Resuspend cells in 300 ul of media and 300 ul of Matrigel
17. Plate 100 ul of cell mixture in each well of 6-well plate
18. Incubate for 30 min at 37 C
19. Add 2 ml of media to each well

### B. T-Cell Expansion Procedure

1. TILs expansion with three conditions:
   i. negative control, no treatment
   ii. add 300 U of IL2 per 1 ml of media (50 μl IL2 per 1 ml of media)
   iii. add 300 U of IL2 and 5 μl of CD3/CD28 beads per 1 ml of media
2. Activate peripheral T-cells: add 500,000 unselected PBMCs to each well containing organoids:
   i. negative control, no treatment
   ii. add 300 U of IL2 per 1 ml of media (50 μl IL2 per 1 ml of media)
   iii. add 300 U of IL2 and 5 μl of CD3/CD28 beads per 1 ml of media

### Example 2

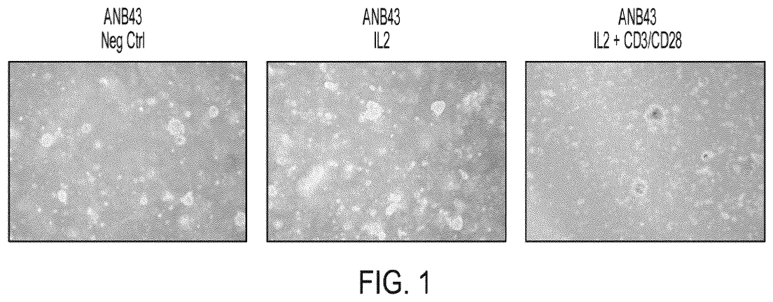

Description of bladder tumor samples to be treated with conditions for organoid-immune cells co-culture:
Tumor ANBCT 42: Large Sample: frozen tumor 30%, RNA later 10%, 10% formalin 10%, organoid 50%. Blood: isolate PBMC using lymphocyte and RBC lysis.
Benign tissue ANBCB42: Large Sample 1.5 cm: frozen 15%, RNA later 5%, 10% formalin 5%, 75% for organoid and TOCRIB
PBMC: 186/2×2/1×10⁴=1.86×10⁶ cells/ml
ANBCB=303×2×10⁴=6.06×10⁶ cells/ml
ANBCT=98×10⁴=9.8×10⁵ cells/ml
ANB43: Small TURBT sample: Tumor split into 3—control 5 μl, IL2, IL2+CD3+CD28. Blood split into 3—control, IL2 3000 U, IL2+CD3+CD28. Reagents were added the next day. FIG. 1

Description of flow cytometry to verify identity of PBMC expanded:

Flow cytometry on ANB43 PMBC-control, IL2, IL2+CD3+CD28 beads. Stained with: 1) unstained and PI only; 2) FITC α-hCD4 (RPA-T4), BV421 α-hCD8 (RPA-78), PerCP/Cy5.5 αhCD3 (Biolegend); 3) PerPC/Cy5.5 αhCD3, APC αhCD137 (4B4-1), PE αhCD107a (H4A-3); 4) BV421 αhCD8, APC αhCD137, PE αhCD107a

Description of flow cytometry experiment on tumor-infiltrating lymphocytes grown in co-culture with organoids from bladder and renal cell carcinoma, FFPE/paraffin sections were also prepared from the same samples:

To prepare co-cultures for FACS analysis (organoids+ T-cells), cells were digested with TryLE and Dispase together at 37° C. for 5 mins. Cells were washed twice with 5 ml of HBSS with 0.2% FBS. Cells were fixed with 4% paraformaldehyde. Cells were washed twice with 5 ml of HBSS with 0.2% FBS. Cells were stored at 4° C. Samples were ANB51, P0 TILs+PBMC; JOB36 TILs+PBMC; ANR1, P0 TILs+PBMC; JOB38 only TILs.

To prepare FFPE for IF/IHC of the following co-cultures (organoids and T-cells) ANB51, P0 TILs+PBMC; ANR1, P0 TILs+PBMCl; JOB36, P0 TILs+PBMC; JOB38 P0 only TILs. 30% of culture were taken and fixed with 10% formaldehyde and stored at 4° C.

Description of RCC sample with normal parenchyma (tumor adjacent) grown in the same co-culture conditions as the tumor:

To establish ANR2 tumor and normal tissue organoids to co-culture with T-cells. Followed co-culture protocol for both tissues. Froze four vials of tumor and two vials of normal. Did FFPE on 1 piece each. Treatments were as follows:

| | | | |
|---|---|---|---|
| PBMC | Negative Control | 6000 IU IL2 | 300 IU IL2 |
| | | | CD3/CD28 |
| Tumor | Negative Control | 6000 IU IL2 | 300 IU IL2 |
| | | | CD3/CD28 |
| Normal Adjacent | Negative Control | 6000 IU IL2 | 300 IU IL2 |
| | | | CD3/CD28 |

Description of exemplary sample treated with anti-PD1 pembrolizumab:

ANB55 P0, reagents added

| Pembrolizumab 10 ul | 6000 IU IL2 | IL2 |
|---|---|---|
| (No response) | Pembrolizumab | Beads 10 ul |
| | (No Response) | Responding ~10-20% killing |
| Negative | IL2 | PBMC + IL2 + Beads |
| | (No Response) | |

### Example 3

The organoid-immune cell co-culture method described herein is shown in FIG. 2.

FIG. 1 shows results of conventional organoid culture of tumor ANB43 generated from a small TURBT sample. Tumor was split into 3 conditions: negative control 5 μl (left panel), IL2 treatment (center panel), IL2+CD3+CD28 treatment (right panel). TURBT=Transurethral resection of bladder tumor. IL2 stimulates T cell expansion. CD3 and CD28 stimulate T cell activation and expansion. T cell activation is a key event in the adaptive immune response and it is vital to the generation of both cellular and humoral immunity.

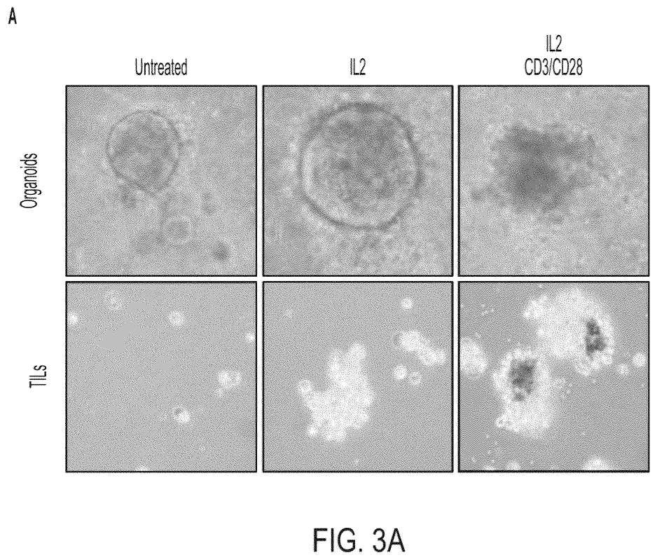

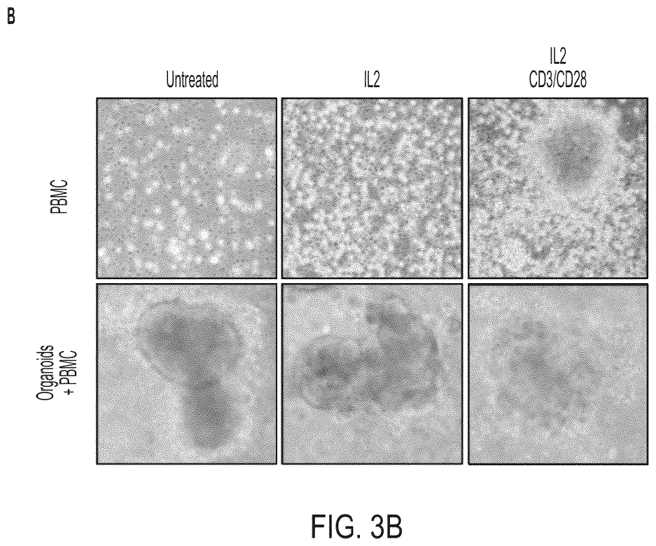

FIGS. 3A-B show conventional organoid culture of bladder cancer. FIG. 3A shows that amplified tumor-infiltrating lymphocytes are able to kill bladder tumor organoids in vitro. Addition of low-dose IL2 (300 IU) increases proliferating lymphocytes but organoids persist in culture. In cultures with T-cell proliferation, addition of CD3/CD28 universally kills 100% of tumor organoids. FIG. 3B shows that treatment of bladder tumor organoids with autologous PBMCs leads to massive tumor organoid killing. There is no difference in killing efficiency with or without added PBMC. N=3 TIL=tumor infiltrating lymphocyte.

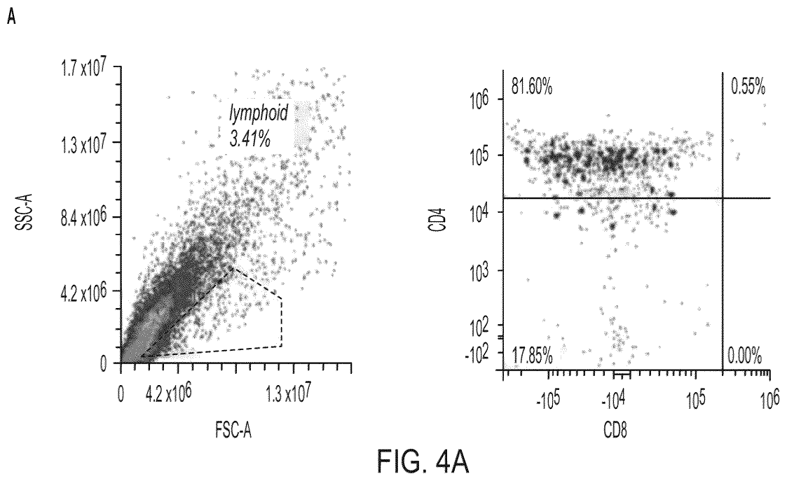

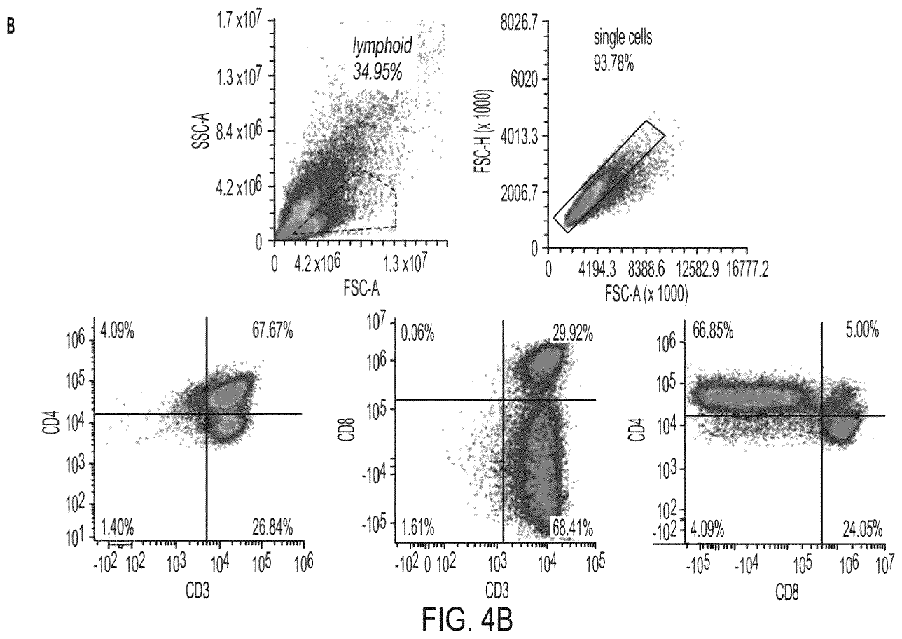

FIGS. 4A-B show a flow cytometry analysis of co-cultured organoids with absolute and relative increase in CD8+ TILs in treated organoid-immune cells co-culture of bladder cancer. FIG. 4A shows composition of immune infiltration in normal bladder organoid culture: 81% are CD4+, CD8− or T-helper cells, 0.55% are CD8+, CD4− or cytotoxic T-cells, 18% are CD8−, CD4−, 4% are double positives. FIG. 4B shows lymphoid composition change with IL2 and CD3/CD28 activation. Relative decrease in CD4+, CD8− or T-helper cells; relative and absolute increase in CD8+, CD4− or cytotoxic T-cells; 5% are CD8−, CD4−; 4% are double positives. Amplified T-lymphocytes have markers of degranulation and cytokine production in organoid-immune cells co-culture.

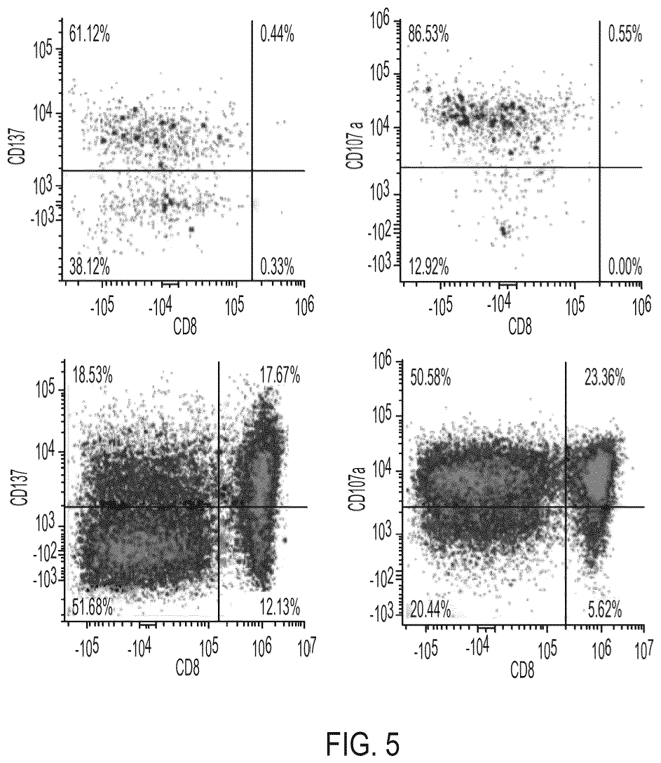

FIG. 5 shows a flow cytometry analysis with an increase in activated CD8+ tumor-infiltrating lymphocytes in treated organoid-immune cells co-culture of bladder cancer. CD137 (4-1BB, TNFR9) is a surface glycoprotein of the TNFR family. CD137 is expressed following activation on T- and NK cells. It is expressed on both activated CD4+ and CD8+ T-cells and enhances proliferation. CD107a is marker of CD8+ T-cell degranulation following stimulation. CD107a correlates with cytokine secretion and NK cell mediated lysis of target cells. Relative and absolute increase in activated CD8+ tumor-infiltrating lymphocytes. Amplified T-lymphocytes have markers of degranulation and cytokine production in organoid-immune cells co-culture. CD137 (4-1BB, TNFR9) is a surface glycoprotein of the TNFR family, expressed following activation on both CD4+ and CD8+ T-cells. CD107a is marker of CD8+ T-cell degranulation and cytokine secretion. Degranulation is a cellular process that releases cytotoxic molecules from granules inside cells. This mechanism is used by cytotoxic T cells (T lymphocytes).

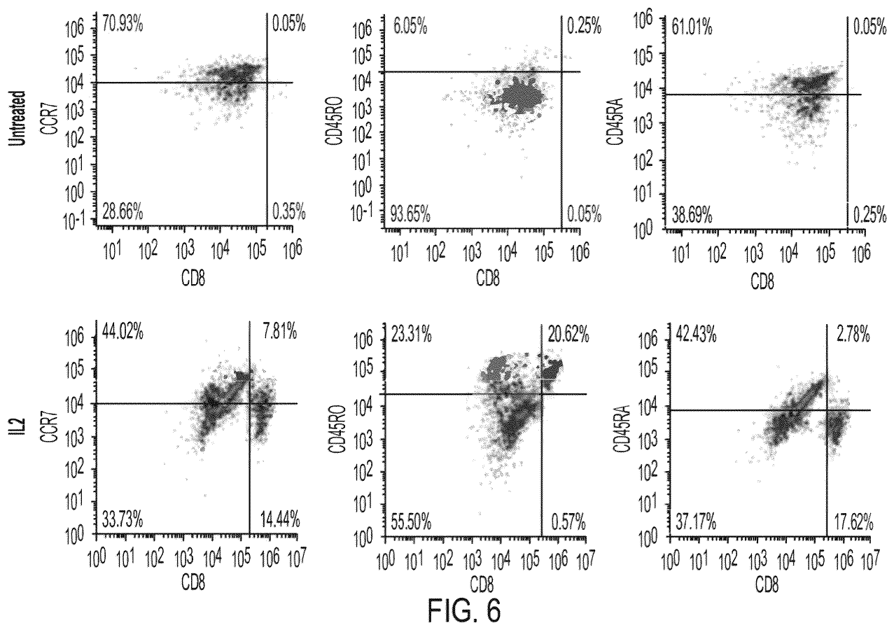

FIG. 6 shows a flow cytometry analysis with CD8+ memory T-cells of bladder cancer. T-lymphocytes are able to form memory subsets in culture. Effector memory T cells (Tem) CD45RA−, CD45RO+, CCR7− specializes in rapidly entering inflamed tissues. Central memory T cells (Tcm) CD45RA−, CD45RO+, CCR7+ express the chemokine receptor CCR7, allowing their re-circulation into the blood and back to lymphoid tissues. The majority of CD8+ memory cells are of the central subtype. A smaller subset of CD8+ cells are effector memory T-cells.

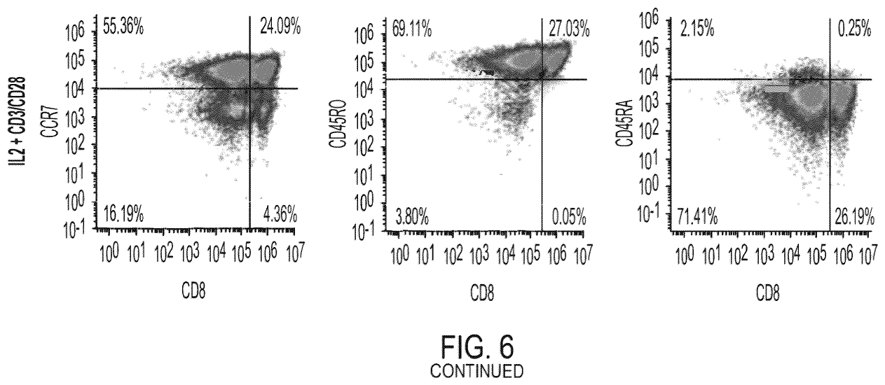

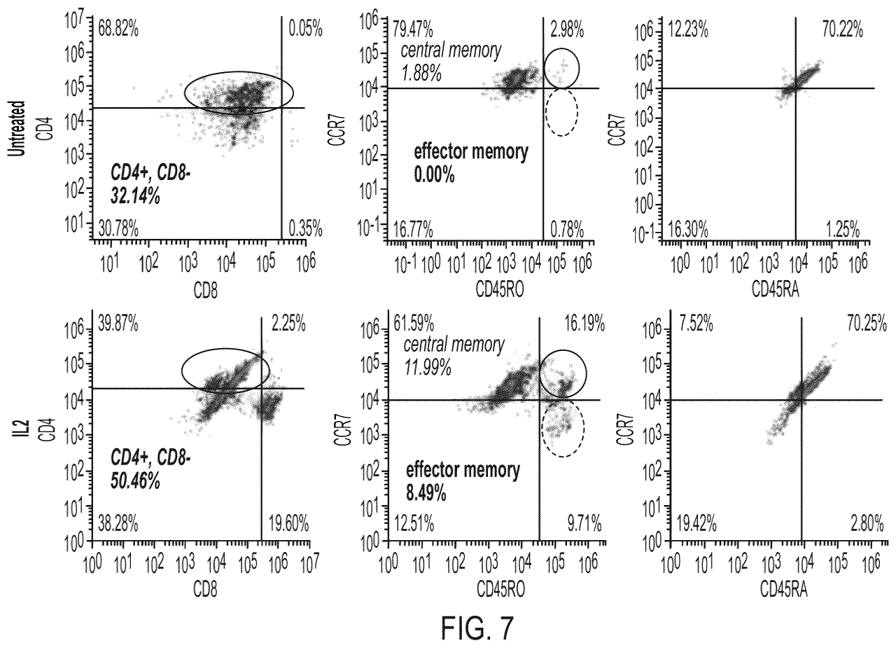

FIG. 7 shows a flow cytometry analysis with CD4+ Memory T-cells of bladder cancer. In unstimulated co-culture, the majority (80%) of tumor-infiltrated CD4+ cells are CD45RO−, CCR7+ which are markers of naïve T-cells. In activated co-culture, the majority (75%) of tumor-infiltrated CD4+ are central memory T-cells (CD45RO+, CCR7+). Effector memory T cells (Tem) (CD45RA−, CD45RO+, CCR7−) make up a smaller (21%) of CD4+ T-cells.

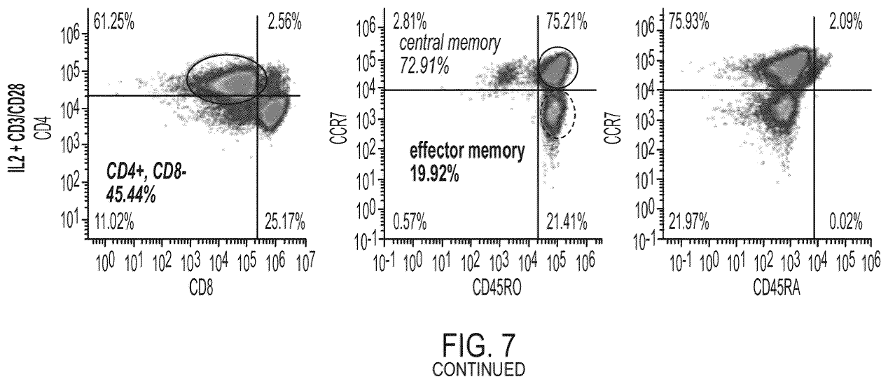

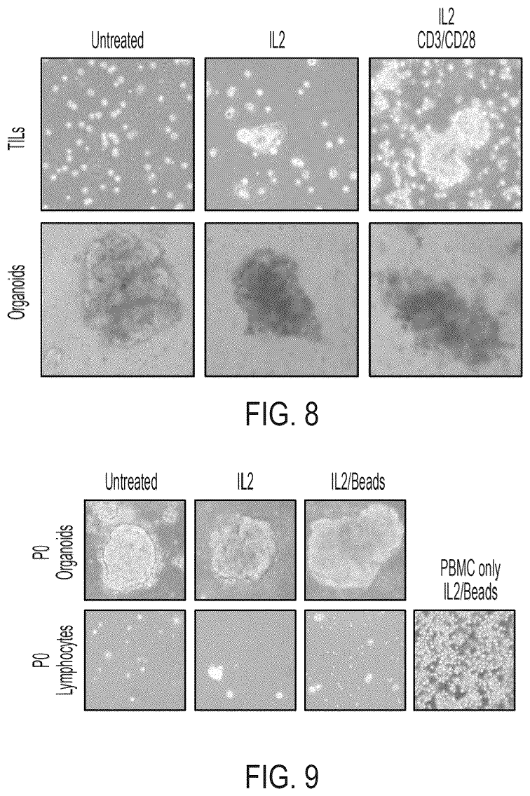

FIG. 8 shows conventional organoid culture in renal cell carcinoma. Addition of low-dose IL2 (300 IU) appears to be sufficient for partial tumor organoid killing in culture. Addition of CD3/CD28 kills 100% of tumor organoids.

FIG. 9 shows that there is a subset of bladder tumors from which no TILs could be amplified. Results for tumor ANB54-HG UC, invasive into lamina propria (Pembrolizumab responsive). Pembrolizumab is a humanized antibody specific for PD-1, a protein on the surface of T cells and B cells. ANB54 is an immune-poor tumor. No amplification of lymphocytes in treated samples but PBMC is able to massively expand. PATIENT: 77M presented with HG Non-muscle-invasive Bladder Cancer (NMIBC) in 2015 with no relapse until March 2021. TURBT was performed on Mar. 22, 2021 followed by gemcitabine and docetaxel (Gem/Doce) instillation. PATHOLOGY: Sample from Mar. 22, 2021 TURBT showed HG papillary urothelial carcinoma, not involving the muscularis propria.

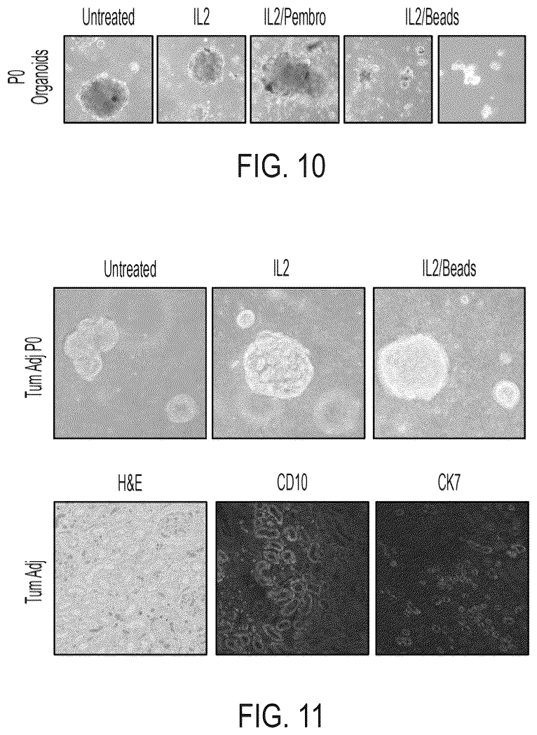

FIG. 10 shows results for tumor ANB57-HG UC, invasive into lamina propria (Pembrolizumab non-responsive). ANB57 is an immune-poor tumor. Tumor organoids did not respond to PD1 blockade with Pembrolizumab. Organoids are still growing in culture post treatment. PATIENT: 66M presented with gross hematuria and multiple tumors seen on CT scans in April 2021. Patient had TURBT on Apr. 19, 2021 and re-TURBT in May 2021. Recommended induction Bacillus Calmette-Guerin (BGC) treatment. PATHOLOGY: Sample from Apr. 19, 2021 TURBT showed HG papillary urothelial carcinoma, with lamina propria invasion. Re-TURBT in May 2021 showed Cis.

FIG. 11 shows results for tumor ANRN2-tumor adjacent organoids. T-cells killed tumor organoids but not normal adjacent tissues. Tumor adjacent organoids survived passaging. P1 and P2 of ANRN2 stock are frozen down.

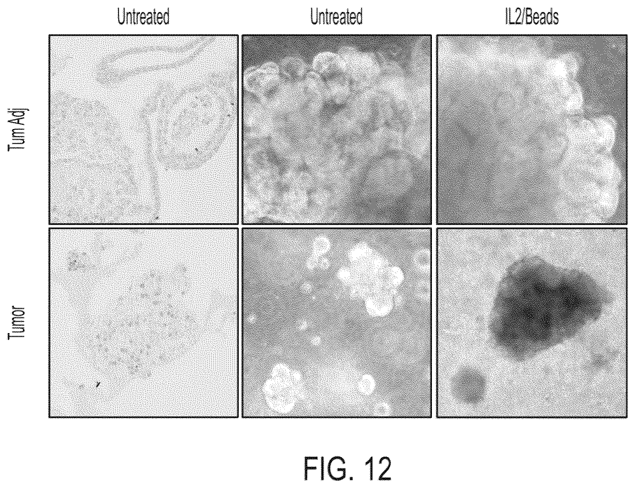

FIG. 12 shows results for tumor JOKT6 and JOKN6. T-cells killed tumor organoids but not normal adjacent tissues. Sample is from a radical nephrectomy of a 10 cm mass, showing ccRCC with extension into the renal sinus and perinephric tissue. Final path pT3aNx. JOKT6, and JOKN6, expanded and have been frozen down, along with TILs and PBMC.

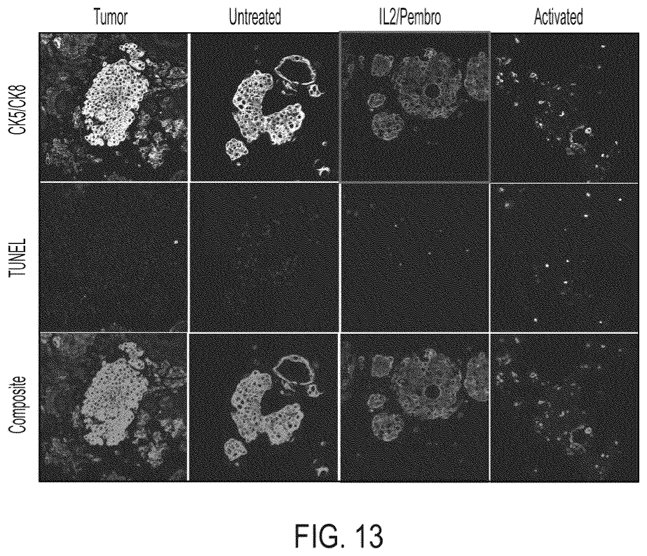

FIG. 13 shows organoid cell death measured by a TUNEL assay. TUNEL staining indicates dying cells. CK5 and CK 8 are luminal cell markers.

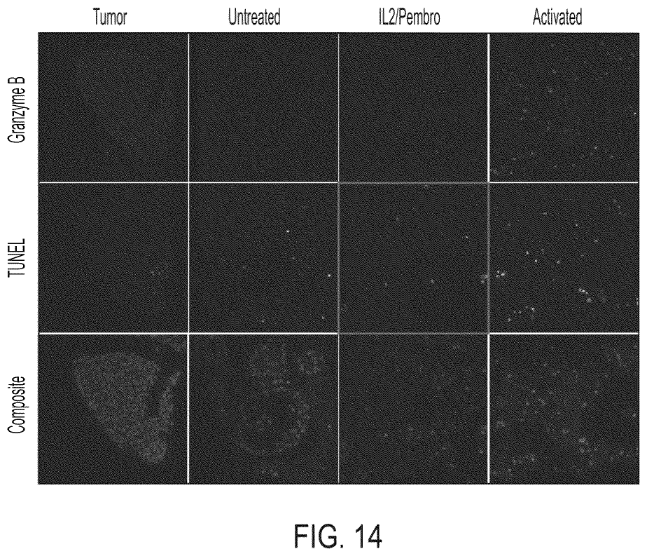

FIG. 14 shows Granzyme B and TUNEL staining. Granzyme B indicates cytotoxic T cell release. Granzyme B expression is increased in treated culture.

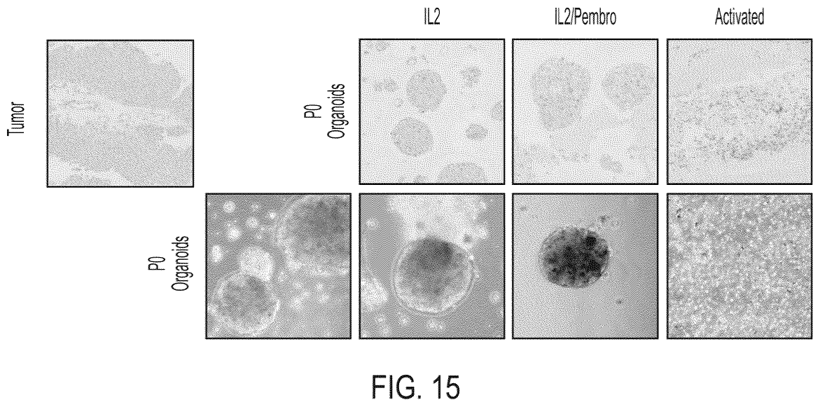

FIG. 15 shows that organoid-immune cells co-culture can be used to test anti-PD1 treatment with Pembrolizumab in vitro. Tumor ANB55 is an immunoresponsive organoid. Tumor organoids had a response to PD1 blockade. PATIENT: 70M presented with 2-3 cm mass on cystoscopy. Patient had TURBT on Apr. 12, 2021 showing HG UC and re-TURBT was negative. BCG induction to be initiated in June 2021. PATHOLOGY: Sample from Apr. 12, 2021 TURBT showed HG papillary urothelial carcinoma, with lamina propria invasion. Re-TURBT in May 2021 was negative for carcinoma.

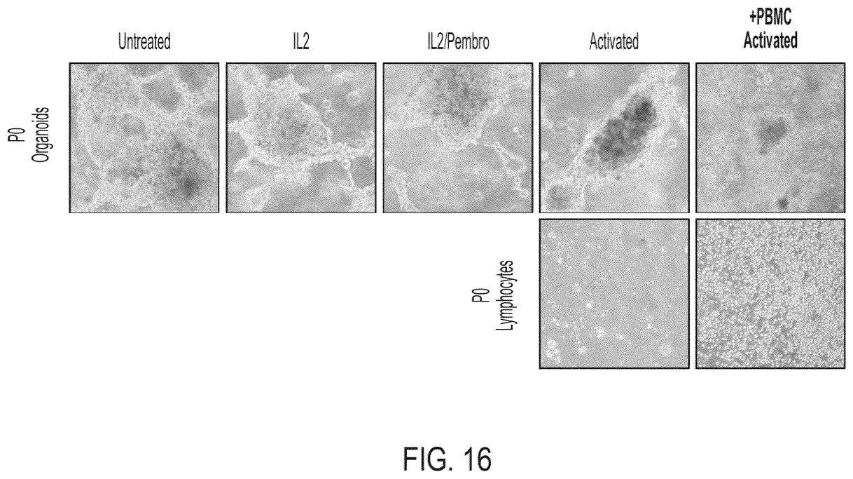

FIG. 16 shows that the patient has tumor-specific T-cells that are present in blood but not in primary tumor. Tumor ANB60 is an immune-non-responsive organoid. Tumor organoids did not respond to PD1 blockade or IL2/beads. Addition of activated PBMC demonstrates that peripheral lymphocytes can be “trained” to recognize and kill tumors. PATIENT: 66F presented with gross hematuria several years ago, recommended cysto but never had it. Developed gross hematuria and abdominal pain in May 2021. PATHOLOGY: Sample from May 17, 2021 TURBT showed non-invasive LG papillary urothelial carcinoma.

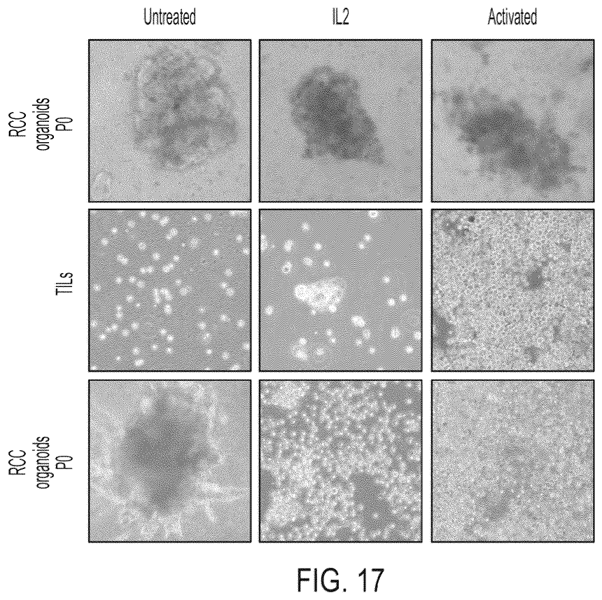

FIG. 17 shows that organoid-immune cells co-culture system can be adopted for treatment of renal cell carcinomas (RCCs).

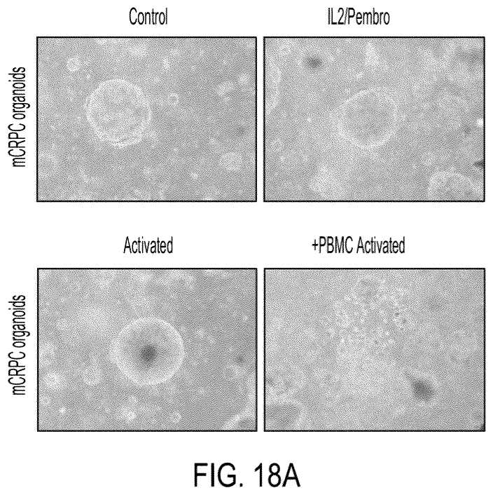

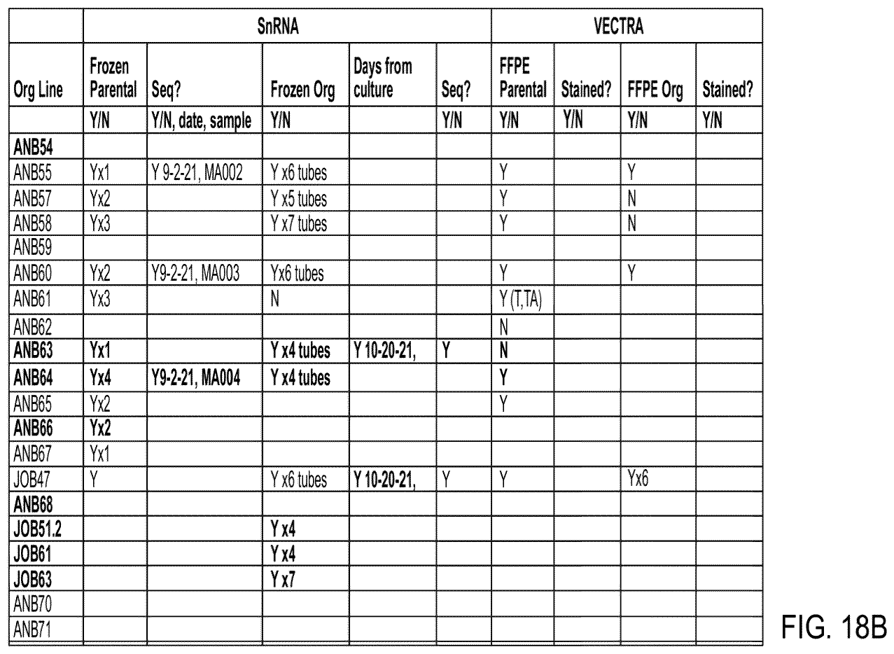

FIGS. 18A-B shows that the organoid-immune cells co-culture disclosed herein can be adopted for castration-resistant prostate cancer. FIG. 18A shows tumor ANP2—a single mCRPC sample. Activation is achieved with treatment with IL2/CD3/CD28. This mCRPC tumor does not respond to checkpoint blockade in vitro, and it also does not have amplified TILs. However, when PBMC is added and activated with IL2/CD3/CD28, there are tumor-specific lymphocytes and can kill tumor organoids. FIG. 18B shows a summary of the organoid-immune cells co-cultures.

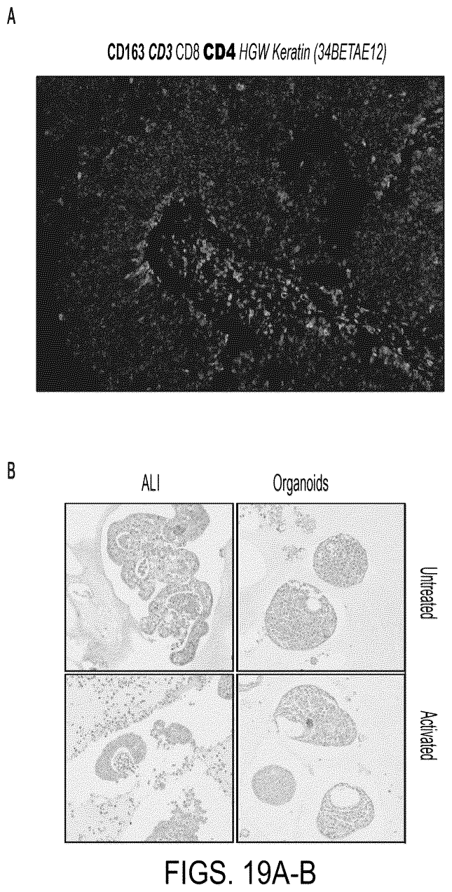

FIGS. 19A-B shows tumor ANB45 NMIBC (papillary-urothelia carcinoma (UC), low grade (LG) and high grade (HG)). FIG. 19A shows Vectra staining. FIG. 19B shows air-liquid interphase culture on left and organoid culture on right.

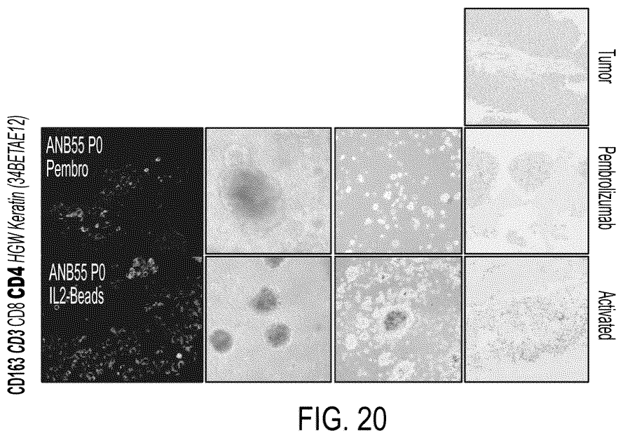

FIG. 20 shows tumor ANB55, NMIBC (HG papillary UC, lamina propria invasion), ANB55 P0, Pembro ANB55 P0.

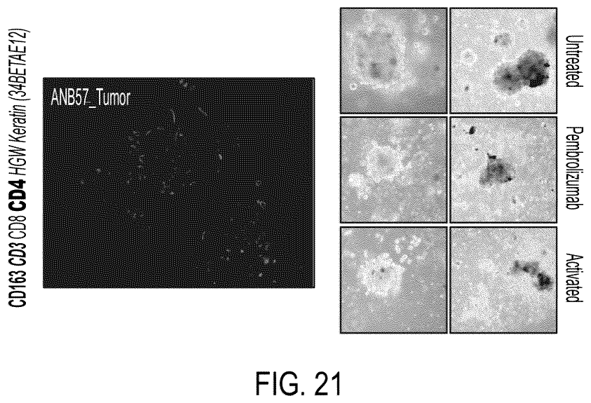

FIG. 21 shows tumor ANB57-high-grade papillary urothelial carcinoma, with laminar propria invasion.

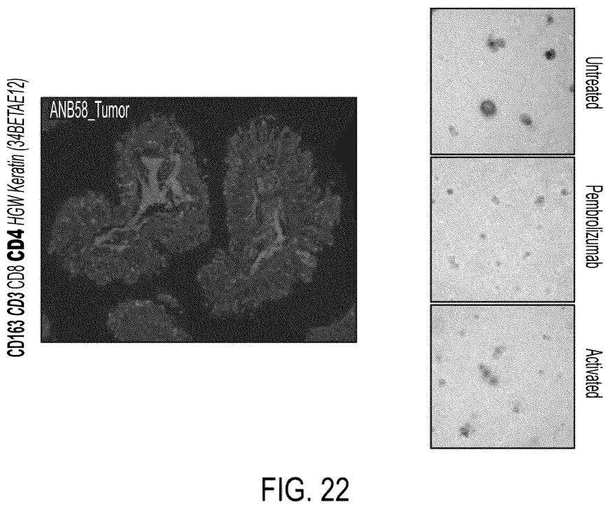

FIG. 22 shows tumor ANB58-upper tract non-invasive (pTa) urothelial carcinoma, Low grade papillary tumor.

### Example 4—Culture Media Protocols

### TIL Rapid Expansion Protocol

1. TILs were put in culture with pooled allogeneic irradiated PBMC feeder cells at a ratio of one TIL to 200 feeders in combination with 6000 IU/mL IL2
2. REP lasts for 14 days
3. Half TIL-CM and half AIM-V (Fisher, Catalog number: 12055083) are used for the first 7 days
4. Only AIM-V is used in the last 7 days

### TIL-CM Media

1. RPMI1640 with GlutaMAX™ (Gibco/Invitrogen)
2. 1× Pen-Strep (Gibco/Invitro-gen)
3. 50 uM 2-mercaptoethanol (Gibco/Invitrogen)
4. 1 mM pyruvate (Gibco/Invitrogen)
5. 10% heat-inactivated human AB Serum (Sigma-Aldrich)
6. 1× of HEPES
7. 6000 IU/mL IL2

### Example 5—Single Nuclei Sequencing of Parental Tumors and Organoid Immune Co-Cultures

Method: Frozen parental tumors and organoids were lysed on ice in lysis buffer. Lysis was stopped with dilution in PBS. Nuclei were precipitated and filtered to obtain a single nuclei suspension. Nuclei morphology were evaluated for quality. Each sample was tagged with a different anti-nucleoporin antibody tag. Nuclei samples were pooled and counted prior to submitting to 10× platform for single nuclei sequencing.

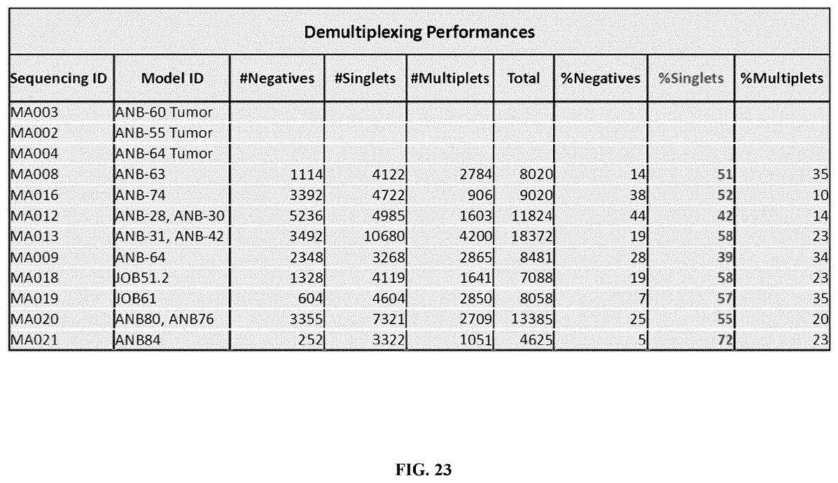

FIG. 23 shows quality control and performance metrics for multiplexed single nuclei sequencing. The majority of nuclei sequenced came from a single cell

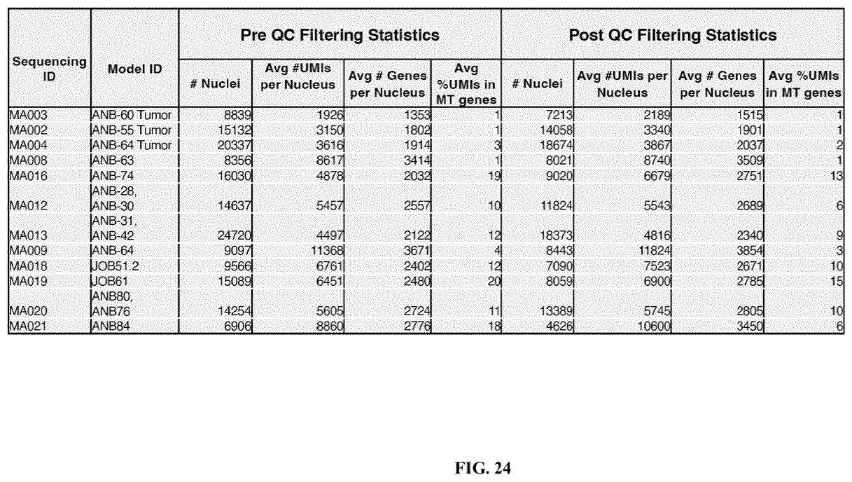

FIG. 24 shows quality control statistics for single nuclei sequencing experiments. Nuclei with >20% mitochondrial genes (MT genes) and nuclei with less than 1500 UMIs (unique molecular identifiers) were filtered out.

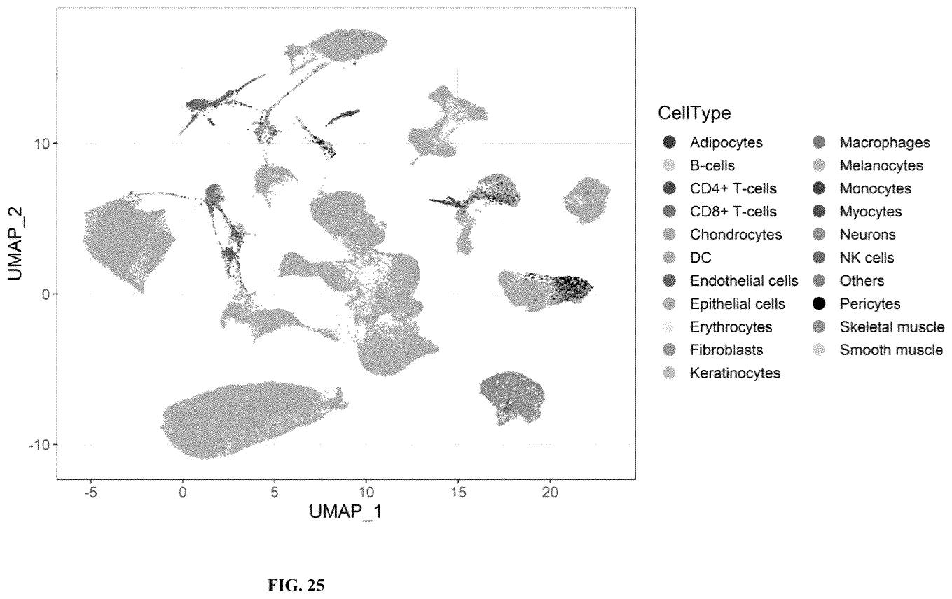

FIG. 25 shows that the single nuclei sequencing disclosed herein identifies multiple subpopulations of cells using singleR references.

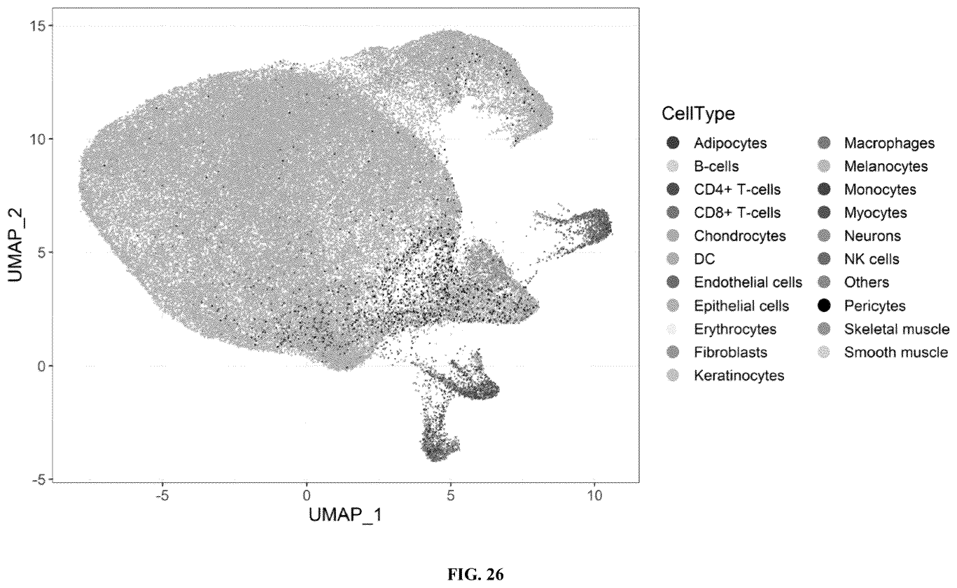

FIG. 26 shows unsupervised clustering of all nuclei after batch correction.

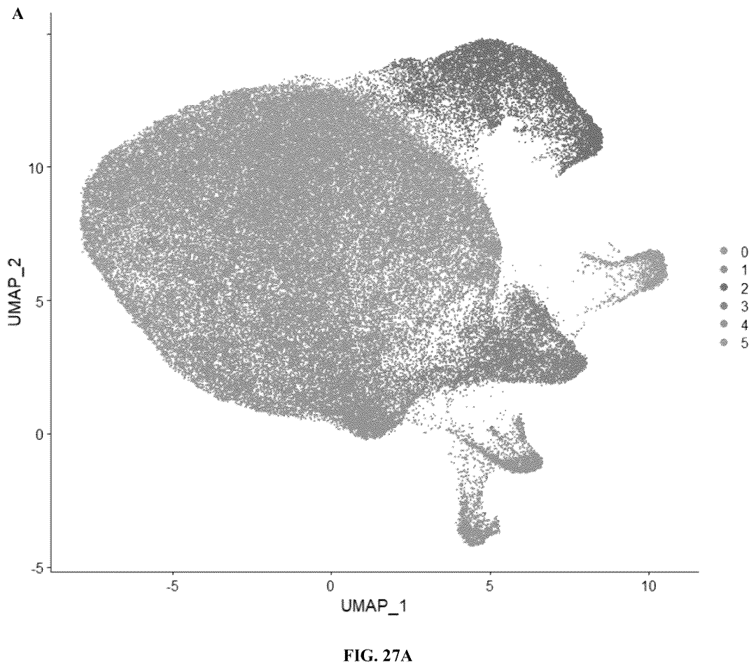

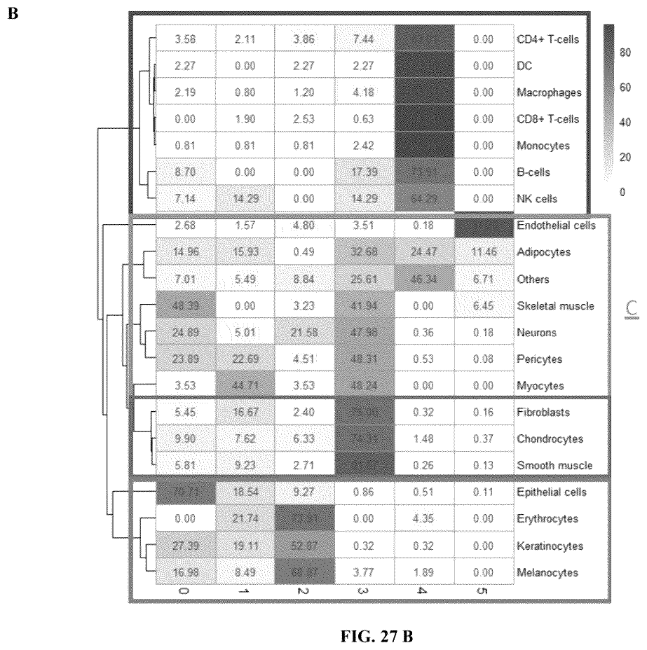

FIG. 27A-B shows clustering of all single nuclei combined. FIG. 27A shows unsupervised clustering of all nuclei after batch correction. FIG. 27B shows the different cell types per cluster in percentages. Distinct cell lineages which can be identified are epithelial and malignant cells (C0-C1-C2, show in blue), primarily cancer associated fibroblasts (C3, shown in green), immune cells of lymphoid and myeloid lineages (C4, shown in purple), and endothelial cells (C5, shown in orange).

FIG. 28 shows the composition of cells in original parental tumors. Most are enriched in tumor cells and cancer-associated fibroblasts. Distinct cell lineages which can be identified are epithelial and malignant cells (top rectangle, show in blue), cancer associated fibroblasts (2^{nd} rectangle from the top, shown in green), immune cells of lymphoid and myeloid lineages (3^{rd} rectangle from the top, shown in purple), and endothelial cells (bottom rectangle, shown in orange).

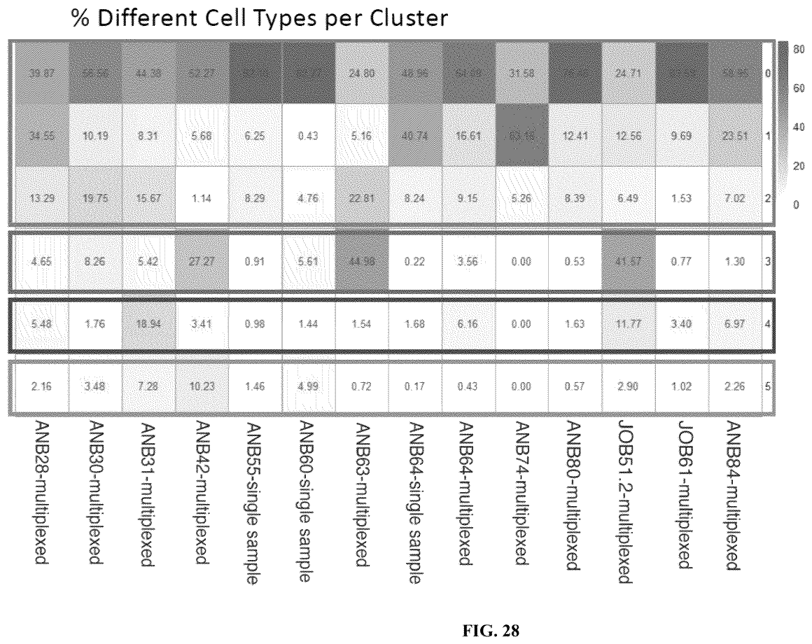

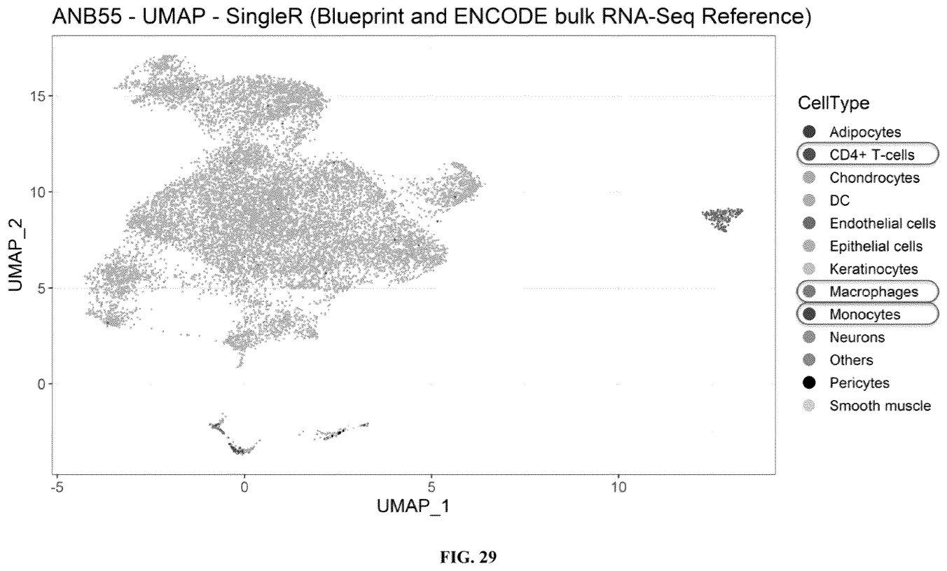

FIG. 29 shows the single nuclei sequencing and cell annotations for tumor sample ANB55. Within the immune compartment, CD4+ T cells, dendritic cells, macrophages, and monocytes could be detected but not CD8+ T cells.

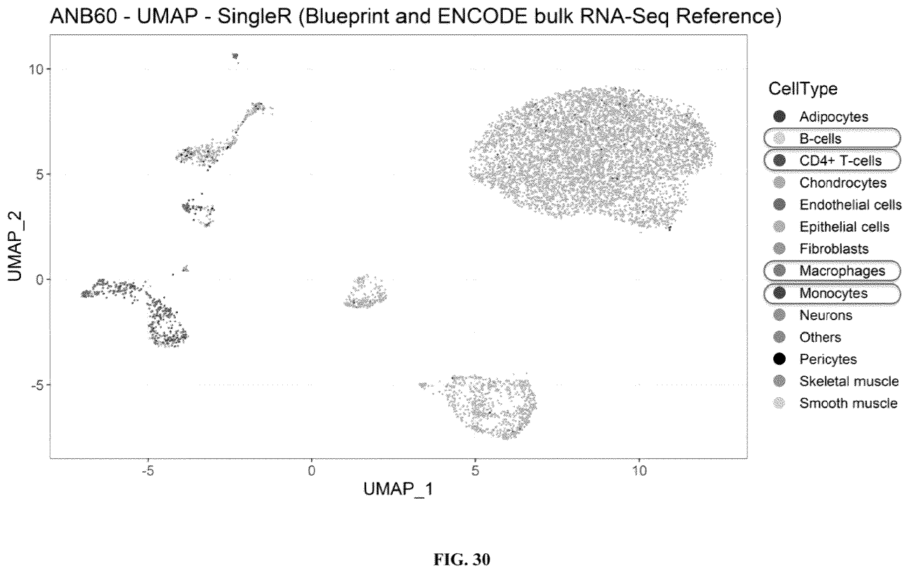

FIG. 30 shows the single nuclei sequencing and cell annotations for tumor sample ANB60. Within immune compartment, CD4+ T cells and macrophages could be detected but not CD8+ T cells.

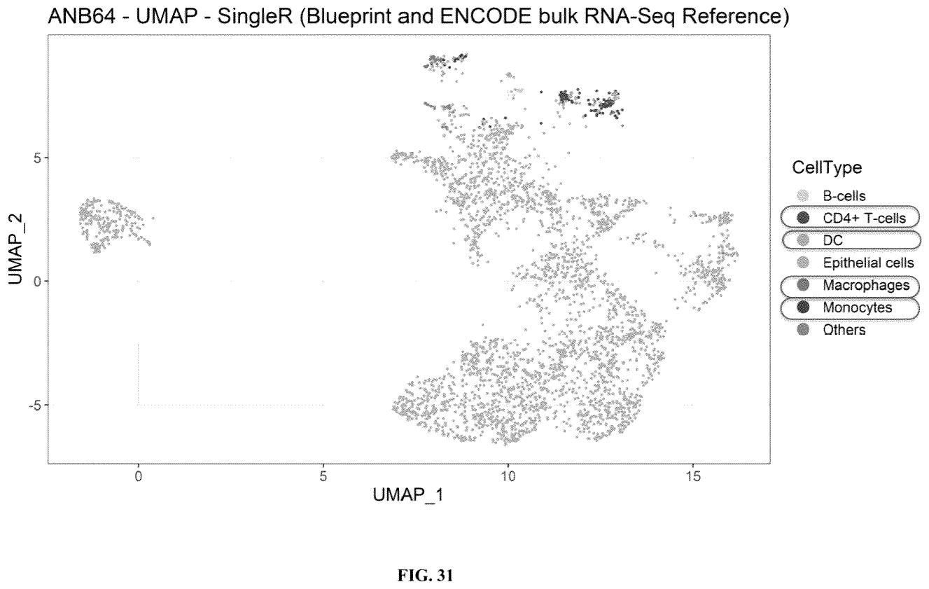

FIG. 31 shows the single nuclei sequencing and cell annotations for tumor sample ANB64. Within immune compartment, CD4+ T cells could be detected but not CD8+ T cells.

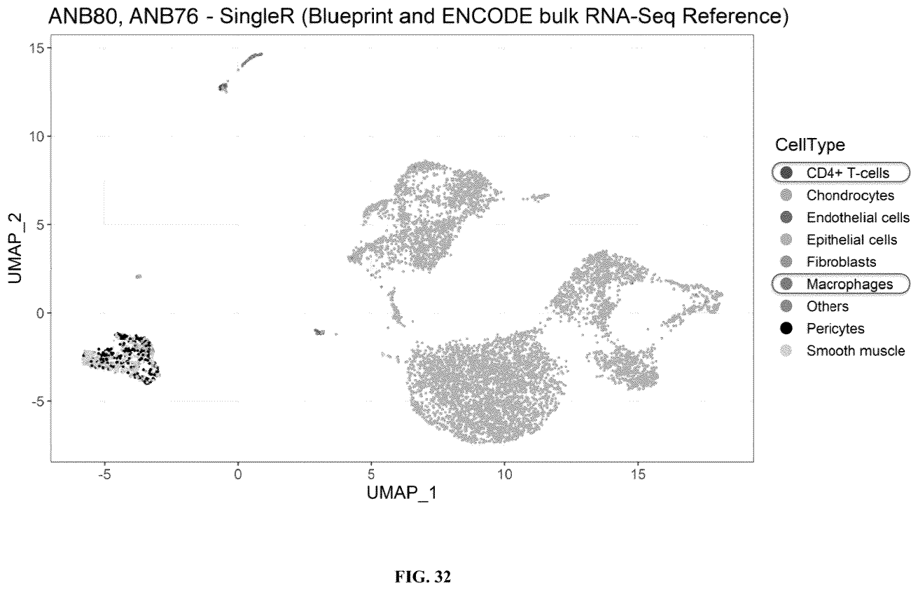

FIG. 32 shows the single nuclei sequencing and cell annotations for tumor sample ANB80 and ANB76. Within immune compartment, CD4+ T cells and macrophages could be detected but not CD8+ T cells.

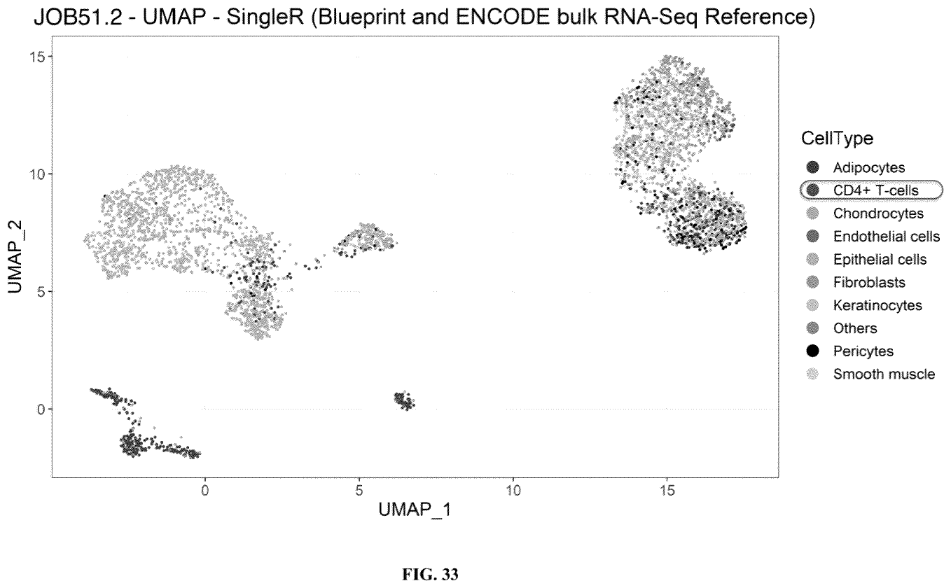

FIG. 33 shows the single nuclei sequencing and cell annotations for tumor sample JOB 51.2. Within immune compartment, CD4+ T cells, macrophages, and monocytes could be detected but not CD8+ T cells.

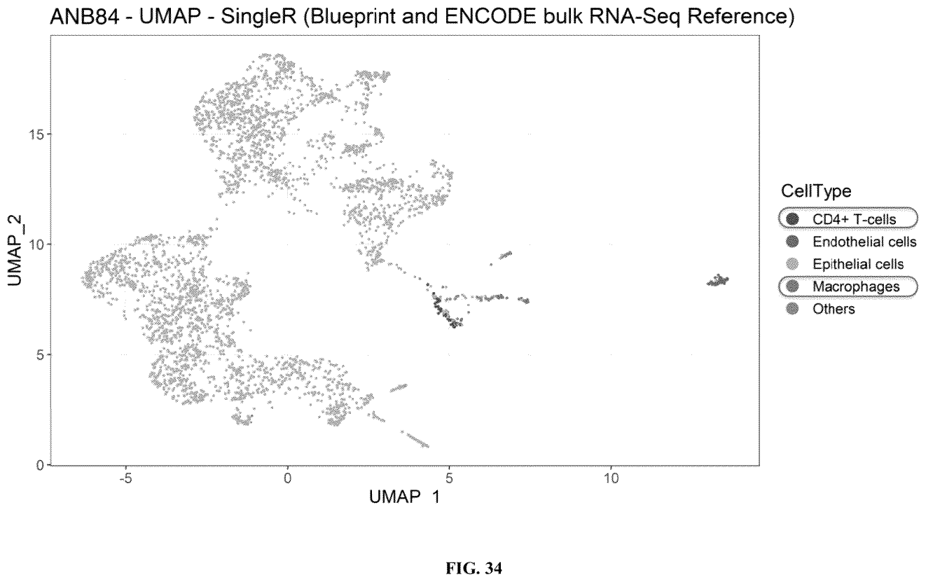

FIG. 34 shows the single nuclei sequencing and cell annotations for tumor sample ANB84. Within immune compartment, CD4+ T cells, macrophages, and monocytes could be detected but not CD8+ T cells.

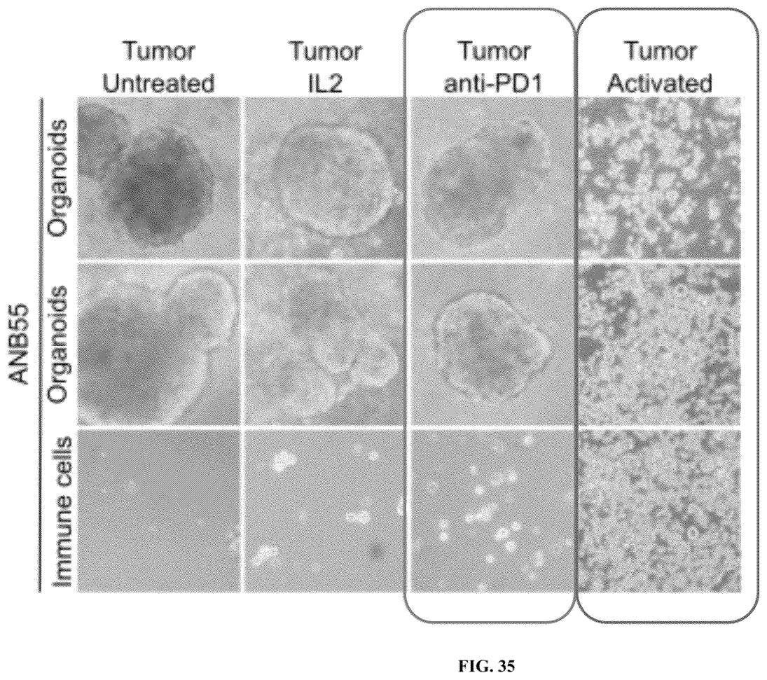

FIG. 35 shows viability assays of tumor ANB55 incubated with various treatments. The majority of bladder tumors lack sufficient CD8+ T cells, but the patient's autologous PBMC could be a source of tumor-specific T-cells. ANB55 does not respond to IL2/Pembrolizumab treatment in vitro. Tumor infiltrating lymphocytes within the tumor mass could be activated and amplified to kill a tumor. Activated treatment means the sample is incubated with IL2 and CD3-CD38 beads.

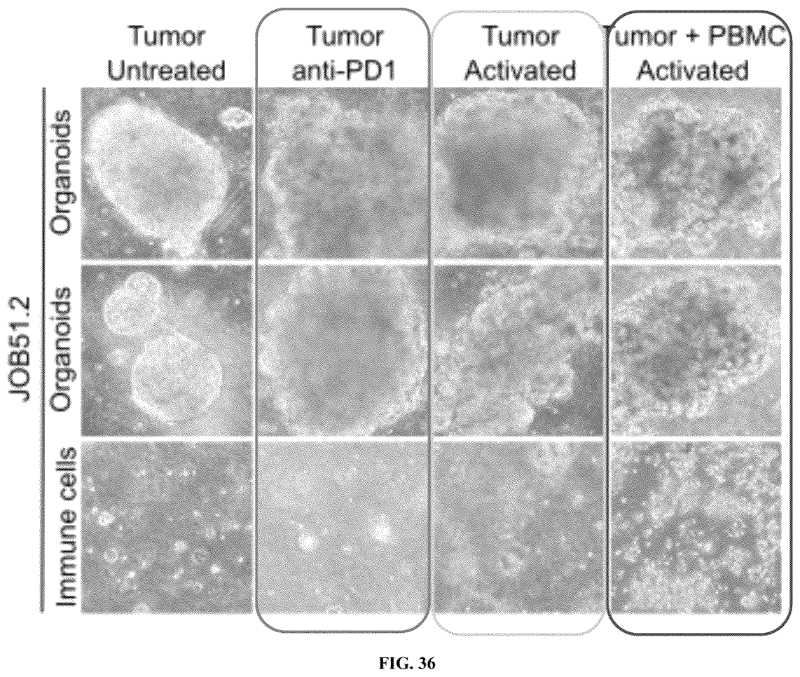

FIG. 36 shows viability assays of tumor JOB51.2 incubated with various treatments. JOB51.2 does not respond to IL2/Pembrolizumab treatment and TILs could not be amplified from the tumor mass. The addition of PBMC leads to tumor killing suggesting the presence of tumor-specific T cells in the patients that do not infiltrate the tumor mass. Activated treatment means the sample is incubated with IL2 and CD3-CD38 beads.

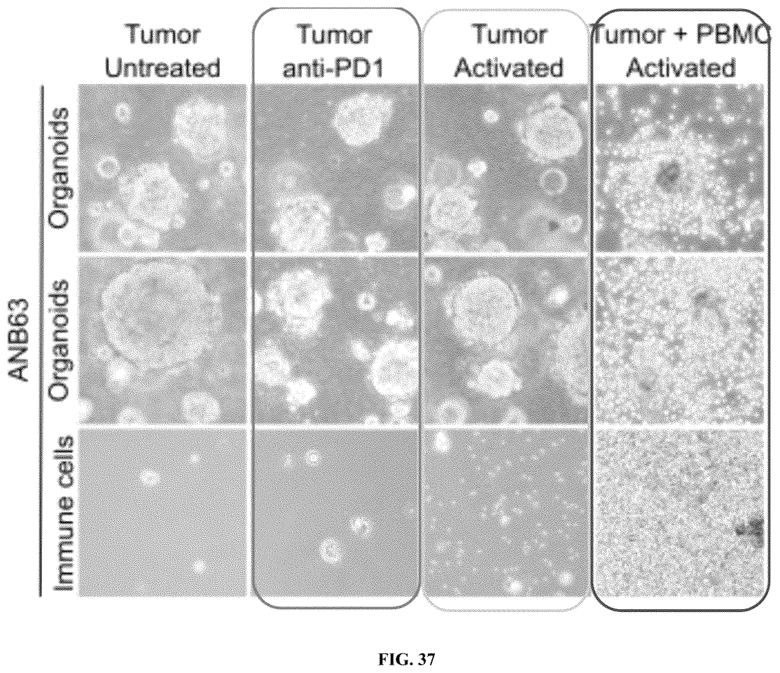

FIG. 37 shows viability assays of tumor ANB63 incubated with various treatments. ANB63 does not respond to IL2/Pembrolizumab treatment and TILs could not be amplified from the tumor mass. The addition of PBMC leads to tumor killing suggesting the presence of tumors specific T cells in the patients that do not infiltrate the tumor mass. Activated treatment means the sample is incubated with IL2 and CD3-CD38 beads.

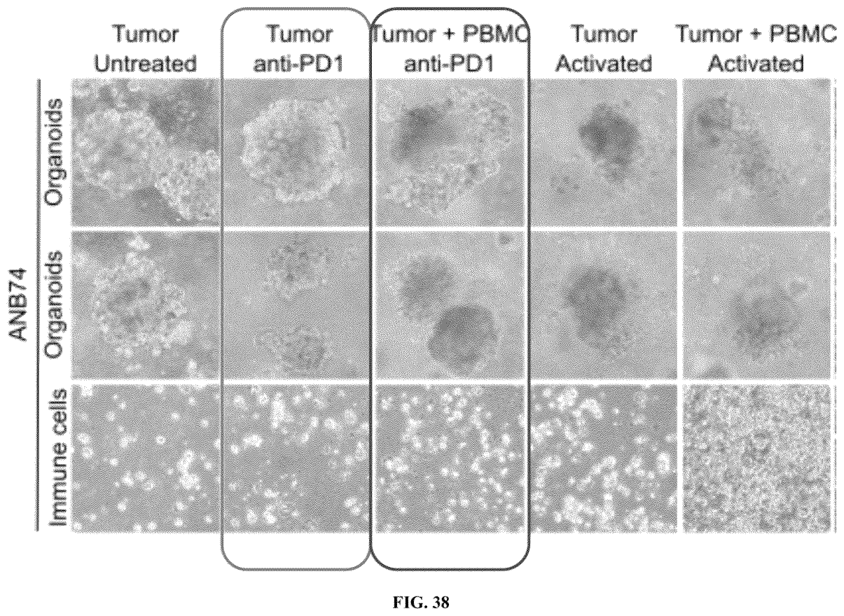

FIG. 38 shows viability assays of tumor ANB74 incubated with various treatments. ANB74 does not respond to IL2/Pembrolizumab treatment alone. The addition of PBMC in addition to IL2/Pembrolizumab was sufficient to improve tumor killing. Activated treatment means the sample is incubated with IL2 and CD3-CD38 beads.

Taken together, the data shows that a majority of bladder cancers do not have CD8+ cytotoxic T cells, which might explain why only 20% of patients respond to immunotherapy.

The data suggests that the content of the tumor mass differs drastically from patient to patient and so is the response to different in vitro treatment conditions.

The data suggests that the organoid-immune co-culture in combination with snRNA/scRNA sequencing can be used to evaluate and potentially inform the best strategy to induce a response in patients. The treatment can be IL2/anti-PD1, IL2/anti-PD1 and PBMC, IL2/CD3-CD28 beads, or IL2/CD3-CD28 beads and PBMCs.

What is claimed is:

1. A method for treating cancer in a subject in need thereof, comprising:
a) producing an organoid-immune cells co-culture from a cancer tissue sample from a subject with cancer;
b) subjecting the organoid-immune cells co-culture to conditions wherein if tumor-infiltrating lymphocytes (TILs) are present within the organoid-immune cells co-culture they are expanded;
c) subjecting the organoid-immune cells co-culture to conditions wherein if TILs are present within the organoid-immune cells co-culture they are activated;
d) determining whether growth of the organoid is inhibited by the activated TILs, wherein: (i) inhibition of growth of the organoid indicates identification of TILs within the organoid-immune cells co-culture and a therapeutically effective amount of the said TILs are administered to the subject, or (ii) lack of inhibition of growth of the organoid indicates absence of TILs within the organoid and a therapeutically effective amount of TILs are not administered to the subject.

2. The method of claim 1, wherein steps b) and c) are performed concurrently.

3. The method of claim 2, wherein if TILs are identified within the organoid-immune cells co-culture, the method further comprises isolating or separating TILs from the organoid-immune cells co-culture.

4. The method of claim 3, wherein the isolated or separated TILs are expanded by culturing the cells in a cell culture medium.

5. The method of claim 4, wherein the isolated or separated TILs are expanded by culturing the cells in a cell culture medium with allogenic irradiated PBMC feeder cells.

6. The method of claim 5, wherein the cell culture medium is a non-Matrigel™ containing T-cell medium.

7. The method of claim 5, wherein the isolated or separated TILs are expanded by culturing the cells for seven days in a mixture of TIL-CM and AIM-V medias.

8. The method of claim 7, wherein the wherein the isolated or separated TILs are further expanded by culturing the cells for an additional seven days in AIM-V medium.

9. The method of claim 1, wherein if TILs are identified within the organoid-immune cells co-culture, the method further comprises isolating or separating TILs from the organoid-immune cells co-culture and expanding the isolated or separated TILs by culturing the cells in a cell culture medium before said TILs are administered to the subject.

10. The method of claim 1, wherein step b) comprises adding cell culture media comprising Interleukin 2 (IL2) to the organoid-immune cells co-culture.

11. The method of claim 1, wherein step c) comprises adding CD3/CD28 beads to the organoid-immune cells co-culture.

12. The method of claim 1, wherein the administration comprises intravenous administration of the TILs.

13. The method of claim 1, wherein if there is a lack of inhibition of growth of the organoid indicating absence of TILs within the organoid-immune cells co-culture, the method further comprises:
e) adding a cell culture media comprising PBMCs, IL2, and CD3/CD28 beads to the organoid-immune cells co-culture;
f) determining whether growth of the organoid is inhibited, wherein: (i) inhibition of growth of the organoid indicates identification of peripheral lymphocytes within the PBMCs that can target the organoid or (ii) lack of inhibition of growth of the organoid indicates absence of peripheral lymphocytes within the PBMCs that can target the organoid.

14. The method of claim 13, wherein the PBMCs are obtained from the subject.

15. The method of claim 13, wherein if peripheral lymphocytes that can target the organoid are identified within the PBMCs, the method further comprises isolating or separating said peripheral lymphocytes from the organoid-immune cells co-culture.

16. The method of claim 15, wherein the isolated or separated peripheral lymphocytes are expanded by culturing the cells in a cell culture medium.

17. The method of claim 16, wherein the isolated or separated peripheral lymphocytes are expanded by culturing the cells in a cell culture medium with allogenic irradiated PBMC feeder cells.

18. The method of claim 17, wherein the cell culture medium is a non-Matrigel™ containing T-cell medium.

19. The method of claim 17, wherein the isolated or separated peripheral lymphocytes are expanded by culturing the cells for seven days in a mixture of TIL-CM and AIM-V medias.

20. The method of claim 19, wherein the wherein the isolated or separated peripheral lymphocytes are further expanded by culturing the cells for an additional seven days in AIM-V medium.

21. The method of claim 13, wherein if peripheral lymphocytes are identified that can target the organoid are identified within the PBMCs, the method further comprises isolating or separating said peripheral lymphocytes from the organoid-immune cells co-culture and expanding the isolated or separated peripheral lymphocytes by culturing the cells in a cell culture medium and administering said peripheral lymphocytes to the subject.

22. The method of claim 1, further comprising evaluating cellular heterogeneity of the organoid-immune cells co-culture or the cancer tissue sample, or both.

23. The method of claim 22, wherein the cellular heterogeneity is evaluated using single nuclei sequencing.

24. The method claim 21, wherein the administration comprises intravenous administration of the peripheral lymphocytes.

25. The method claim 1, wherein the cancer is a bladder cancer, a breast cancer, a prostate cancer, a pancreatic cancer, a kidney tumor, a renal cell carcinoma, a lung tumor, a colorectal cancer, uterine cancer, a thyroid tumor, or a brain cancer.

26. The method of claim 1, wherein the cancer is bladder cancer, prostate cancer, or renal cell carcinoma.

27. The method of claim 1, wherein producing an organoid-immune cells co-culture from a cancer tissue sample from a subject with cancer comprises:
a) dissociating the sample of tissue;
b) contacting the dissociated tissue with a Matrigel™ solution and plating in a cell culture support, wherein the Matrigel™ solution comprises cell culture medium and Matrigel™ and wherein the Matrigel™ solution forms a matrix;
c) incubating the culture wherein the dissociated tissue forms organoid-immune cells co-culture.

28. A pharmaceutical composition comprising a therapeutically effective amount of the isolated or separated TILs of claim 3.

29. A pharmaceutical composition comprising a therapeutically effective amount of the isolated or separated peripheral lymphocytes of claim 15.

30. A method for identifying a compound for administration to a subject with cancer, the method comprising:
a) producing an organoid-immune cells co-culture from a cancer tissue sample from a subject with cancer;
b) contacting the organoid-immune cells co-culture with a test compound;
c) determining whether growth of the organoid is inhibited in the presence of the test compound, as compared to growth of the organoid in the absence of the test compound;
wherein inhibition of growth of the organoid indicates identification of the test compound for administration to said subject.

31. A method for treating cancer in a subject in need thereof, comprising:
a) obtaining a cancer tissue sample from a subject with cancer;
b) dissociating the sample of tissue;
c) contacting the dissociated tissue with a Matrigel™ solution and plating in a cell culture support, wherein the Matrigel™ solution comprises cell culture medium and Matrigel™ and wherein the Matrigel™ solution forms a matrix;
d) incubating the culture of (c), wherein the dissociated tissue forms organoid-immune cells co-culture
e) contacting the organoid-immune cells co-culture with a test compound; and
f) determining whether growth of the organoid is inhibited in the presence of the test compound, as compared to growth of the organoid in the absence of the test compound, wherein the test compound is administered to the subject if growth of the organoid is inhibited in the presence of the test compound.
